(19) United States
(12) Patent Application Publication
(10) Pub. No.: US 2008/0200461 A1
(43) Pub. Date: Aug. 21, 2008

(54) MODULATORS OF ACETYL-COENZYME A CARBOXYLASE AND METHODS OF USE THEREOF

(75) Inventors: Richard Anderson, Palo Alto, CA (US); Takeo Hokama, Mountain View, CA (US); Shy-Fuh Lee, Sunnyvale, CA (US); Rafael Oey, San Jose, CA (US); Tedd Elich, Durham, NC (US); Steven Breazeale, Durham, NC (US)

Correspondence Address: MYERS BIGEL SIBLEY & SAJOVEC, PO BOX 37428, RALEIGH, NC, 27627, US

(73) Assignee: Cropsolution, Inc.

(21) Appl. No.: 12/033,925

(22) Filed: Feb. 20, 2008

Related U.S. Application Data
(60) Provisional application No. 60/890,643, filed on Feb. 20, 2007.

(51) Int. Cl. A61K 31/5377; C07D 239/72; C07D 413/06; A61K 31/5375; C07D 265/30; A61K 31/497

(52) U.S. Cl. 514/235.8; 544/316; 544/116; 544/162; 514/252.17; 514/237.8

(57) ABSTRACT

The present invention provides compounds of formula I:

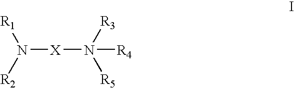

along with methods of use thereof for the control of agricultural pests, particularly fungal pests, weedy pests and insect pests, as well as use as pharmaceuticals, particularly the treatment of obesity, metabolic syndrome, atherosclerosis, cardiovascular disease and insulin resistance, e.g., type II or adult-onset diabetes as well as fungal pathogens of humans and animals

### RELATED APPLICATIONS

The present application claims the benefit, under 35 U.S.C. §119(e), of U.S. Provisional Application No. 60/890,643, filed on Feb. 20, 2007, the disclosure of which is incorporated by reference herein in its entirety.

This invention was made with Government support under grant number DK68962 from the National Institutes of Health. The Government has certain rights to this invention.

### FIELD OF THE INVENTION

The present invention concerns substituted amines and ammonium salts, compositions thereof, and methods of use thereof for the control of agricultural pests, particularly fungal pests, weedy pests and insect pests, as well as use as pharmaceuticals, particularly the treatment of obesity, metabolic syndrome, atherosclerosis, cardiovascular disease and insulin resistance, e.g., type II or adult-onset diabetes as well as fungal pathogens of humans and animals.

### BACKGROUND OF THE INVENTION

This invention relates to the identification of Acetyl CoA carboxylase (ACC) modulators, agricultural and pharmaceutical compositions containing such modulators, and the use of such modulators. Modulators of ACC can have applications in both the agricultural and pharmaceutical areas. In agricultural applications, such compositions could be utilized as fungicides as well as other crop protectants such as insecticides and herbicides. In pharmaceutical applications, such compositions could be utilized to treat, for example, obesity, metabolic syndrome, atherosclerosis, cardiovascular disease and insulin resistance, e.g., type II or adult-onset diabetes, in human or animal subjects, and may also have utility as human and animal antifungal compounds.

Acetyl CoA carboxylase (ACC) catalyzes the first committed step in fatty acid biosynthesis and therefore is an essential enzyme in most organisms. This makes ACC an attractive agrochemical target and ACC is chemically validated as a fungicide, herbicide, and insecticide target as described in more detail in Elich et al., US Patent Application Publication No. 2004/0086994. Additionally, ACC plays a crucial role in the metabolism of fatty acids in mammals and therefore is an important target for drug development against obesity, diabetes and other diseases (Abu-Elheiga, L. et al., Science 291, 2613-2616 (2001); Alberts, A. W., and Vagelos, P. R. Acyl-CoA Carboxylases. In The Enzymes, P. D. Boyer, ed. (New York, Academic Press), pp. 37-82 (1972); Cronan Jr., J. E., and Waldrop, G. L., Prog Lipid Res 41, 407-435 (2002); Harwood Jr., H. J. et al., J Biol Chem 278, 37099-37111 (2003); Wakil, S. J. et al., Ann Rev Biochem 52, 537-579 (1983); Zhang, H. et al., Crystal structure of the carboxyltransferase domain of acetyl-coenzyme A carboxylase in complex with CP-640186. Structure 12, 1683-1691 (2004a); Zhang, H. et al., Proc Natl Acad Sci USA 101, 5910-5915 (2004b); Zhang, H. et al., Science 299, 2064-2067 (2003)). ACCs catalyze the carboxylation of acetyl-CoA to produce malonyl-CoA. Mammals have two isoforms of ACC, ACC1 and ACC2. ACC1 is present in the cytosol of liver and adipose tissues and controls the committed step in the biosynthesis of long-chain fatty acids. In comparison, ACC2 is associated with the outer membrane of mitochondria in the heart and muscle. Its malonyl-CoA product is a potent inhibitor of carnitine palmitoyltransferase I, which facilitates the transport of long-chain acyl-CoAs into the mitochondria for oxidation (McGarry, J. D. et al., Eur J Biochem 244, 1-14 (1997); Ramsay, R. R. et al., Biochim Biophys Acta 1546, 21-43 (2001)). The importance of ACCs for drug discovery is underscored by the observations that mice lacking ACC2 have elevated fatty acid oxidation, reduced body fat and body weight (Abu-Elheiga, L. et al., Proc Natl Acad Sci USA 100, 10207-10212 (2003); Lenhard, J. M. et al., Advanced Drug Delivery Reviews 54, 1199-1212 (2002)).

Soraphen A was originally isolated from the culture broth of Soiangium cellulosum, a soil dwelling myxobacterium, for its potent antifungal activity (Gerth, K., et al., J Antibiot (Tokyo) 47, 23-31 (1994); Gerth, K. et al., J Biotech 106, 233-253 (2003)). Use of soraphen in ACCase assays has been more fully described in Elich et al., US Patent Application Pub. No. 2004/0086994. This polyketide natural product contains an unsaturated 18-membered lactone ring, an extracyclic phenyl ring, two hydroxyl groups, three methyl groups, and three methoxy groups (Bedorf, N. et al., Liebigs Ann Chem 9, 1017-1021 (1993); Ligon, J. et al., Gene 285, 257-267 (2002)) (FIG. 2A). There is also a 6-membered ring within the macrocycle formed by a hemiketal between the C3 carbonyl and C7 hydroxyl (FIG. 2A). Soraphen A has demonstrated strong promise as a broad-spectrum fungicide against various plant pathogenic fungi (Pridzun, L., Untersuchungen zum Wirkungsmechanismus von Soraphen A, Technical University of Braunschweig (1991)). Genetic and biochemical studies show that soraphen A is a potent inhibitor of the BC domain of eukaryotic ACCs (Gerth et al., supra (1994; 2003); Pridzun, supra (1991); Pridzun, L. et al., Inhibition of fungal acetyl-CoA carboxylase: a novel target discovered with the myxobacterial compound soraphen. In Antifungal agents, G. K. Dixon, L. G. Copping, and D. W. Hollomon, eds. (Oxford, UK, BIOS Scientific Publishers Ltd.), pp. 99-109 (1995); Vahlensieck, H. F. et al., U.S. Pat. No. 5,641,666 (1997); Vahlensieck, H. F. et al., Curr Genet 25, 95-100 (1994)), with K_{d} values of about 1 nM. Additionally, soraphen inhibits both mammalian ACC isoforms and has been demonstrated to have pharmacological properties consistent with the potential to treat obesity and diabetes (Gubler, M. and Mizrahi, J., PCT WO03011867). In comparison, the compound has no effect on bacterial BC subunits (Behrbohm, H., Acetyl-CoA Carboxylase aus Ustilago maydis. Reinigung, Charakterisierung und Intersuchungen zur Inhibierung durch Soraphen A, Technical University of Braunschweig (1996); Weatherly, S. C. et al., Biochem J 380, 105-110 (2004)).

We have used recombinant biotin carboxylase domains in a high-throughput soraphen competition binding assay to identify novel small molecules that bind to sites that overlap with the soraphen binding site and that inhibit ACC activity. These molecules have use as fungicides, herbicides, insecticides, and pharmaceuticals for diabetes, obesity and related metabolic disorders as well as antifungal applications in humans and animals.

### SUMMARY OF THE INVENTION

A first aspect of the present invention is a compound of formula I:

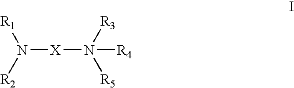

wherein:

R₁ and R₂ are taken independently from H, alkyl, alkenyl, alkynyl, haloalkyl, haloalkenyl, haloalkynyl, COR₆, COOR₆, CONR₇R₈, SO₂NR₇R₈, SO₂R₉, alkoxyalkyl, alkylthioalkyl, aryl, arylalkyl or aryloxyalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,

or R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted (e.g., 1-3 times) by halogen, alkyl, haloalkyl, alkoxyalkyl, haloalkoxyalkyl, arylalkyl, ═O, ═S, and optionally fused to another 5- or 6-membered ring;

R₃ and R₄ are taken separately from alkyl, alkenyl, alkynyl, haloalkyl, haloalkenyl, haloalkynyl, CH₂COR₆, CH₂COOR₆, CH₂CONR₇R₈, O, aryl, arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,

or R₃ and R₄ may be taken together to form a 5- or 6-membered ring comprised of 3 to 5 carbon atoms, 1 to 2 nitrogen atoms, 0 to 1 oxygen atom, and 0 to 1 sulfur atom, optionally substituted (e.g., 1-3 times) by halogen, alkyl, haloalkyl, alkoxyalkyl, haloalkoxyalkyl, arylalkyl, ═O, ═S, and optionally fused to another 5- or 6-membered ring;

R₅ may be a non-bonded pair of electrons, alkyl, alkenyl, alkynyl, haloalkyl, haloalkenyl, haloalkynyl; arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂;

when R₅ is alkyl, alkenyl, alkynyl, haloalkyl, haloalkenyl, haloalkynyl; arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂, the N-atom to which R₃, R₄, and R₅ are attached will carry a positive charge and be associated with anion Y⁻, where Y may be halogen, carboxylate, sulfonate or any other suitable counterion;

R₆, R₇, R₈ and R₉ are independently H, alkyl, haloalkyl, alkoxyalkyl, haloalkoxyalkyl, alkylthioalkyl, haloalkylthioalkyl, aryl or arylalkyl optionally substituted (e.g., 1-3 times) with halogen, alkyl, haloalkyl, alkoxy, haloalkoxy, alkylthio, haloalkylthio, CN, aryl, arylalkyl, aryloxy, arylalkoxy, arylalkylthio, alkylene, haloalkylene, alkylenedioxy, haloalkylenedioxy, wherein said aryl, arylalkyl, aryloxy, arylalkoxy, arylalkylthio is optionally substituted (e.g., 1-3 times) with independently selected halogen, alkyl, haloalkyl, alkoxy, or haloalkoxy;

X is a bridge group.

The compounds and compositions of the present invention are useful as crop protection agents to combat or prevent fungal infestations, or to control other pests such as weeds, insects, or acarids that are harmful to crops.

A second aspect of the present invention is a composition for controlling and preventing plant pathogenic microorganisms comprising, in combination, an active compound or combination of compounds as described herein together with a suitable carrier.

A third aspect of the present invention is a method of controlling or preventing infestation of cultivated plants by pathogenic microorganisms, comprising applying an active compound or combination of compounds as described herein to said plants, parts thereof or the locus thereof in an amount effective to control said microorganisms.

A further aspect of the present invention is a method of controlling or preventing infestation of technical materials by pathogenic microorganisms, comprising applying an active compound as described herein to said technical materials, parts thereof or the locus thereof in an amount effective to control said microorganisms.

A further aspect of the present invention is a method of treating a fungal infection in a subject in need thereof, comprising administering an active compound as described herein to said subject in an amount effective to treat said fungal infection.

A still further aspect of the present invention is the use of an active compound as described herein for the preparation of a composition (e.g., an agricultural formulation, a pharmaceutical formulation) for carrying out a method as described herein (e.g., an agricultural treatment as described herein, the treatment of technical materials as described herein, the treatment of a fungal infection in a subject as described herein, the treatment or prevention of disease or disorders in humans are as described herein).

A still further aspect of the invention is a method of treating metabolic syndrome, insulin resistance syndrome or obesity in a subject in need of such treatment, comprising administering to said subject a treatment-effective amount of a compound as described herein.

The foregoing and other objects and aspects of the present invention are explained in greater detail below.

### DETAILED DESCRIPTION OF THE PREFERRED EMBODIMENTS

“Alkyl” as used herein refers to a saturated hydrocarbon radical which may be straight-chain or branched-chain (for example, ethyl, isopropyl, t-amyl, or 2,5-dimethylhexyl) or cyclic (for example cyclobutyl, cyclopropyl or cyclopentyl) and contains from 1 to 24 carbon atoms. This definition applies both when the term is used alone and when it is used as part of a compound term, such as “haloalkyl” and similar terms. In some embodiments, preferred alkyl groups are those containing 1 to 4 carbon atoms, which are also referred to as “lower alkyl.” In some embodiments preferred alkyl groups are those containing 5 or 6 to 24 carbon atoms, which may also be referred to as “higher alkyl”.

“Alkenyl,” as used herein, refers to a straight or branched chain hydrocarbon containing from 2 to 24 carbons and containing at least one carbon-carbon double bond formed by the removal of two hydrogens. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl and the like. “Lower alkenyl” as used herein, is a subset of alkenyl and refers to a straight or branched chain hydrocarbon group containing from 1 to 4 carbon atoms.

“Alkynyl,” as used herein, refers to a straight or branched chain hydrocarbon group containing from 2 to 24 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and the like. “Lower alkynyl” as used herein, is a subset of alkyl and refers to a straight or branched chain hydrocarbon group containing from 1 to 4 carbon atoms.

“Alkoxy” refers to an alkyl radical as described above which also bears an oxygen substituent which is capable of covalent attachment to another hydrocarbon radical (such as, for example, methoxy, ethoxy and t-butoxy).

“Alkylthio” as used herein refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Aryl” or “aromatic ring moiety” refers to an aromatic substituent which may be a single ring or multiple rings which are fused together, linked covalently or linked to a common group such as an ethylene or methylene moiety. The aromatic rings may each contain heteroatoms and hence “aryl” encompasses “heteroaryl” as used herein. Representative examples of aryl include, azulenyl, indanyl, indenyl, naphthyl, phenyl, tetrahydronaphthyl, biphenyl, diphenylmethyl, 2,2-diphenyl-1-ethyl, thienyl, pyridyl and quinoxalyl. “Aryl” means substituted or unsubstituted aryl unless otherwise indicated and hence the aryl moieties may be optionally substituted (e.g., 1-3 times) with halogen atoms, or other groups such as nitro, carboxyl, alkoxy, phenoxy and the like. The substituents on vicinal (adjacent) carbon atoms of an aromatic ring may fuse to form a two ring compound; such vicinal substituents include (C2 to C4 or C6) alkylene, (C2 to C4 or C6) haloalkylene, (C2 to C4 or C6) alkylenedioxy and/or (C2 to C4 or C6) haloalkylenedioxy, e.g., methylenedioxy, ethylenedioxy and/or difluoromethylenedioxy. Additionally, the aryl radicals may be attached to other moieties at any position on the aryl radical which would otherwise be occupied by a hydrogen atom (such as, for example, 2-pyridyl, 3-pyridyl and 4-pyridyl).

“Heteroaryl” means a cyclic, aromatic hydrocarbon in which one or more carbon atoms have been replaced with heteroatoms. If the heteroaryl group contains more than one heteroatom, the heteroatoms may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom S may be substituted with one or two oxo groups, which may be shown as ═O. In addition, the substituents on vicinal (adjacent) carbon atoms of an aromatic ring may fuse to form a two ring compound; such vicinal substituents include (C2 to C4 or C6) alkylene, (C2 to C4 or C6) haloalkylene, (C2 to C4 or C6) alkylenedioxy and/or (C2 to C4 or C6) haloalkylenedioxy, e.g., methylenedioxy, ethylenedioxy and/or difluoromethylenedioxy.

“Agriculturally acceptable salt” means a salt, the cation of which is known and accepted in the art, for the formation of salts for agricultural or horticultural use. Preferably the salts are water-soluble.

“Cyano” as used herein refers to a —CN group.

“Halo” or “halogen,” as used herein, refers to —Cl, —Br, —I or —F.

“Haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Hydroxy,” as used herein, refers to an —OH group.

“Nitro,” as used herein, refers to a —NO₂ group.

“Oxy,” as used herein, refers to a —O— moiety.

“Thio,” as used herein, refers to a —S— moiety.

“Organic base” as used herein includes but is not limited to triethylamine, triisobutylamine, trioctylamine, triisodecylamine, diethanolamine, triethanolamine, pyridine, morpholine, and mixtures thereof. A preferred category of organic bases is organic amines.

“Inorganic base” as used herein includes but is not limited to sodium carbonate, sodium bicarbonate, potassium carbonate, and mixtures thereof.

“Inert solvent” as used herein includes any suitable inert solvent, such as tetrahydrofuran, N-methylpyrrolidone, dimethylformamide, toluene, dimethyl ether, methyl t-butyl ether and dioxane, methylene chloride, chloroform, 1,2-dichloroethane, and mixtures thereof.

“Protic solvent” as used herein may be any suitable protic solvent, including but not limited to methanol, ethanol, isopropanol, n-butanol, ethylene glycol, methyl Cellosolve, ethyl Cellosolve, cyclohexanol, glycerol, diethylene glycol, triethanolamine, polyethylene glycol, sec-butanol, n-propanol and tert-butanol.

“Optionally substituted” as used herein refers to a substituent that is either unsubstituted or has one or more (e.g., 1-3 times) additional substituents substituted thereon.

The disclosures of all US Patent references cited herein are to be incorporated herein in their entirety as if fully set forth.

2. Compounds. The compounds of this invention are represented by the structure I:

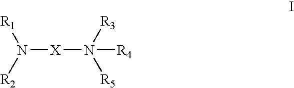

wherein

R₁ and R₂ are taken independently from H, alkyl, alkenyl, alkynyl, haloalkyl, haloalkenyl, haloalkynyl, COR₆, COOR₆, CONR₇R₈, SO₂NR₇R₈, SO₂R₉, alkoxyalkyl, alkylthioalkyl, aryl, arylalkyl or aryloxyalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,

or R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted (e.g., 1-3 times) by halogen, alkyl, haloalkyl, alkoxyalkyl, haloalkoxyalkyl, arylalkyl, ═O, ═S, and optionally fused to another 5- or 6-membered ring;

R₃ and R₄ are taken separately from alkyl, alkenyl, alkynyl, haloalkyl, haloalkenyl, haloalkynyl, CH₂COR₆, CH₂COOR₆, CH₂CONR₇R₈, O, aryl, arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,

or R₃ and R₄ may be taken together to form a 5- or 6-membered ring comprised of 3 to 5 carbon atoms, 1 to 2 nitrogen atoms, 0 to 1 oxygen atom, and 0 to 1 sulfur atom, optionally substituted (e.g., 1-3 times) by halogen, alkyl, haloalkyl, alkoxyalkyl, haloalkoxyalkyl, arylalkyl, ═O, ═S, and optionally fused to another 5- or 6-membered ring;

R₅ may be a non-bonded pair of electrons, alkyl, alkenyl, alkynyl, haloalkyl, haloalkenyl, haloalkynyl; arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂;

when R₅ is alkyl, alkenyl, alkynyl, haloalkyl, haloalkenyl, haloalkynyl, arylalkyl or heteroarylalkyl optionally substituted (e.g., 1-3 times) with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂, the N-atom to which R₃, R₄, and R₅ are attached will carry a positive charge and be associated with anion Y⁻, where Y may be halogen, carboxylate, sulfonate or any other suitable counterion;

R₆, R₇, R₈ and R₉ are independently H, alkyl, haloalkyl, alkoxyalkyl, haloalkoxyalkyl, alkylthioalkyl, haloalkylthioalkyl, aryl or arylalkyl optionally substituted (e.g., 1-3 times) with halogen, alkyl, haloalkyl, alkoxy, haloalkoxy, alkylthio, haloalkylthio, CN, aryl, arylalkyl, aryloxy, arylalkoxy, arylalkylthio, alkylene, haloalkylene, or alkylenedioxy, haloalkylenedioxy, wherein said aryl, arylalkyl, aryloxy, arylalkoxy, arylalkylthio is optionally substituted (e.g., 1-3 times) with independently selected halogen, alkyl, haloalkyl, alkoxy, or haloalkoxy;

X is a bridge group (e.g., alkyl, aryl, heteroaryl) consisting of 2 to 8 C-atoms, 0 to 1 N-atom, 0 to 1 O-atom and 0 to 1 S-atom optionally substituted (e.g., 1-3 times) with halogen, alkyl, haloalkyl, hydroxy, alkoxy, haloalkoxy, alkylthio, cyano, acyl, alkylsulfinyl, alkylsulfonyl, or NO₂, etc.

In some embodiments of the invention, X can comprise, for example, X^{a}—X^{b}—X^{c}, wherein X^{a} and X^{c} can be a covalent bond or C1-C2 alkyl, which can be unsubstituted or substituted with the same substituents as described for X above, and X^{b} can be, for example, an alkyl, aryl, or heteroaryl group consisting of 2-8 C-atoms, 0-1 N-atom, 0-1 O-atom and 0-1 S-atom optionally substituted (e.g., 1-3 times) with halogen, alkyl, haloalkyl, hydroxy, alkoxy, haloalkoxy, alkylthio, cyano, acyl, alkylsulfinyl, alkylsulfonyl, or NO₂, etc.

In some embodiments, X^{c} can be coupled to R³ by a linking group L, as in a compound of Ia

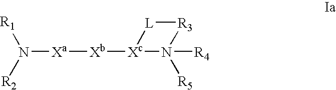

where L can be a covalent bond to R³, (C₂ to C₄ or C₆) alkylene and/or (C₂ to C₄ or C₆) haloalkylene.

The N-containing ring (which may be a fused ring when R³ and R⁴ also form a ring as described above) formed from the linking of X^{c} and R³ via L in compounds of Formula Ia can include heterocycloalkyl, and heteroaryl.

In still further embodiments, X^{b} can be taken alone to be a ring comprised of 3 to 6 carbon atoms, 0 to 1 nitrogen atom, 0 to 1 oxygen atom, and 0 to 1 sulfur atom.

### Methods of Making.

Compositions of generic structure I which are positively charged may be prepared by the alkylation of an amine II with an appropriate halide (Q=Cl, Br, or I) or sulfonate ester (Q=OSO₂R′) III in an inert solvent such as tetrahydrofuran, DMF, or NMP at a temperature range of 0-80° C.

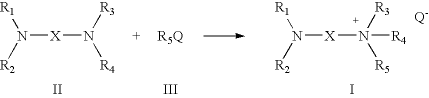

When R₅ is a nonbonded pair of electrons, I=II.

Compositions II may be prepared by generally known methodology, including alkylation, reductive amination, acylation, sulfonylation, and ring closure.

### Exemplary Compounds.

Compounds of the invention that are especially useful for the control of fungal pathogens are those in which:

R₁ and R₂ are taken independently from alkyl, COR₆, COOR₆, CONR₇R₈, SO₂R₉, or arylalkyl optionally substituted (e.g., 1-3 times) with halogen, alkyl, haloalkyl, alkoxy, haloalkoxy,

or R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring;

R₃ and R₄ are taken separately from alkyl, arylalkyl or heteroarylalkyl optionally substituted (e.g., 1-3 times) with halogen, alkyl, haloalkyl, alkoxy, haloalkoxy,

or R₃ and R₄ may be taken together to form a 5- or 6-membered ring comprised of 3 to 5 carbon atoms, 1 to 2 nitrogen atoms, 0 to 1 oxygen atom, and 0 to 1 sulfur atom, optionally substituted (e.g., 1-3 times) by halogen, alkyl, haloalkyl, alkoxyalkyl, haloalkoxyalkyl, arylalkyl, ═O, ═S, and optionally fused to another 5- or 6-membered ring;

R₅ may be a non-bonded pair of electrons, alkyl, arylalkyl or heteroarylalkyl optionally substituted (e.g., 1-3 times) with halogen, alkyl, haloalkyl, alkoxy, haloalkoxy,

when R₅ is alkyl, arylalkyl or heteroarylalkyl optionally substituted (e.g., 1-3 times) with halogen, alkyl, haloalkyl, alkoxy, haloalkoxy, the N-atom to which R₃, R₄, and R₅ are attached will carry a positive charge and be associated with anion Y⁻, where Y may be halogen, carboxylate, sulfonate or any other suitable counterion;

R₆, R₇, R₈ and R₉ are independently H, alkyl, haloalkyl, alkoxyalkyl, haloalkoxyalkyl, alkylthioalkyl, haloalkylthioalkyl, aryl or arylalkyl optionally substituted (e.g., 1-3 times) with halogen, alkyl, haloalkyl, alkoxy, haloalkoxy, alkylthio, haloalkylthio, CN, aryl, arylalkyl, aryloxy, arylalkoxy, arylalkylthio wherein said aryl, arylalkyl, aryloxy, arylalkoxy, arylalkylthio is optionally substituted (e.g., 1-3 times) with independently selected halogen, alkyl, haloalkyl, alkoxy, or haloalkoxy;

X is a bridge group (e.g., alkyl, aryl, heteroaryl) consisting of 2 to 8 C-atoms, 0 to 1 N-atom, 0 to 1 O-atom and 0 to 1 S-atom optionally substituted (e.g., 1-3 times) with halogen, alkyl, haloalkyl, hydroxy, alkoxy, haloalkoxy, alkylthio, cyano, acyl, alkylsulfinyl, alkylsulfonyl, or NO₂, etc.

In some embodiments of the invention, X can comprise, for example, X^{a}—X^{b}—X^{c}, wherein X^{a} and X^{c} can be a covalent bond or C1-C2 alkyl, which can be unsubstituted or substituted with the same substituents as described for X above, and X^{b} can be, for example, an alkyl, aryl, or heteroaryl group consisting of 2-8 C-atoms, 0-1 N-atom, 0-1 O-atom and 0-1 S-atom optionally substituted (e.g., 1-3 times) with halogen, alkyl, haloalkyl, hydroxy, alkoxy, haloalkoxy, alkylthio, cyano, acyl, alkylsulfinyl, alkylsulfonyl, or NO₂, etc.

In some embodiments, X^{c} can be coupled to R³ by a linking group L, as in a compound of Ia

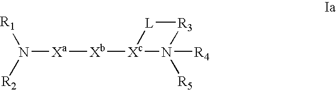

where L can be a covalent bond to R³, (C₂ to C₄ or C₆) alkylene, and/or (C₂ to C₄ or C₆) haloalkylene.

The N-containing ring (which may be a fused ring when R³ and R⁴ also form a ring as described above) formed from the linking of X^{c} and R³ via L in compounds of Formula Ia can include heterocycloalkyl, and heteroaryl.

In still further embodiments, X^{b} can be taken alone to be a ring comprised of 3 to 6 carbon atoms, 0 to 1 nitrogen atom, 0 to 1 oxygen atom, and 0 to 1 sulfur atom.

Examples of compounds of the present invention include, but are not limited to, the following:

| Compound | |
|---|---|
| Number | Structure |
| 1 | 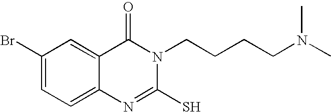 |
| 2 | 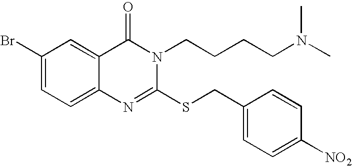 |
| 3 | 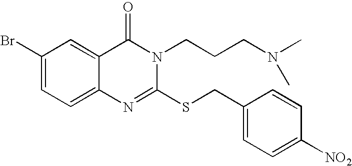 |
| 4 | 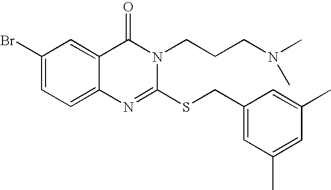 |
| 5 | 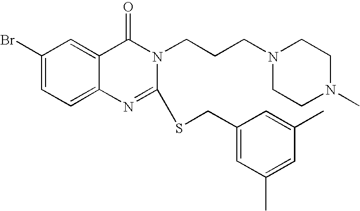 |
| 6 | 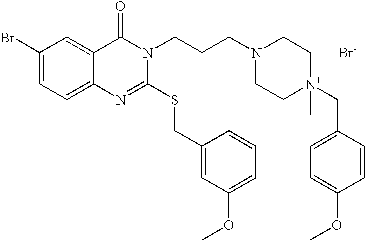 |
| 7 | 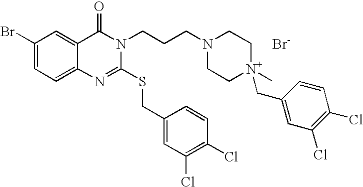 |
| 8 | 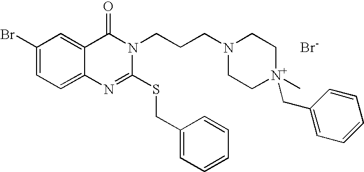 |
| 9 | 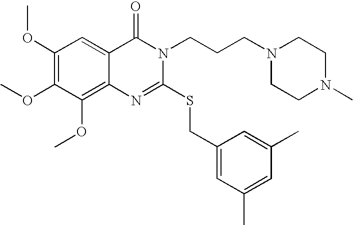 |
| 10 | 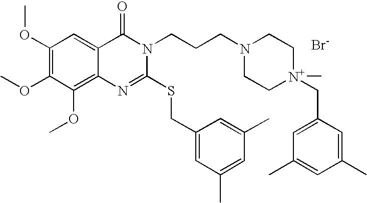 |
| 11 | 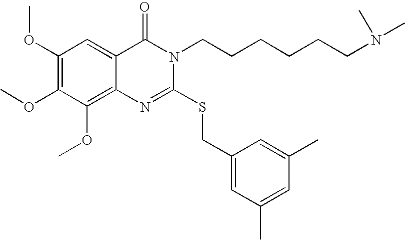 |
| 12 | 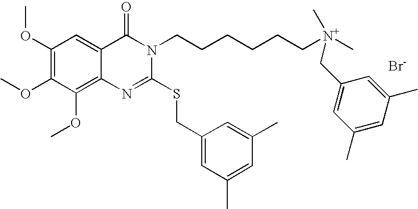 |
| 13 | 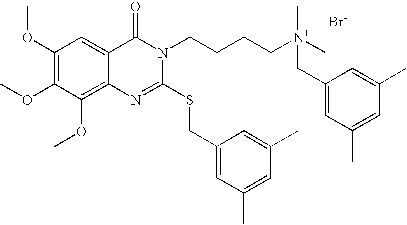 |
| 14 | 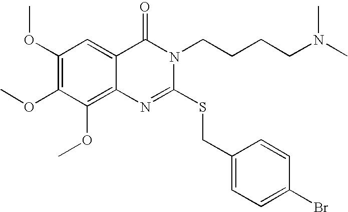 |
| 15 | 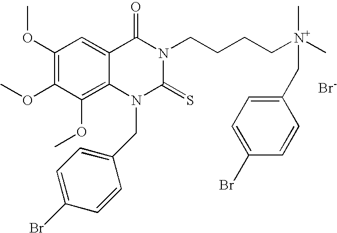 |
| 16 | 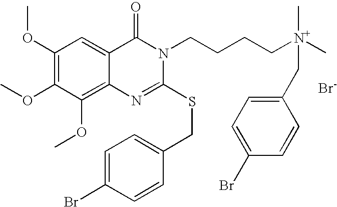 |
| 17 | 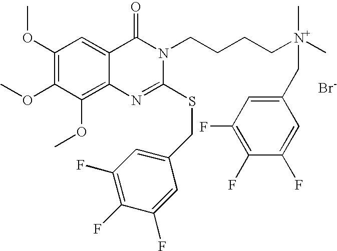 |
| 18 | 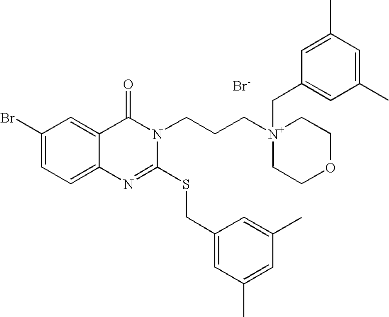 |
| 19 | 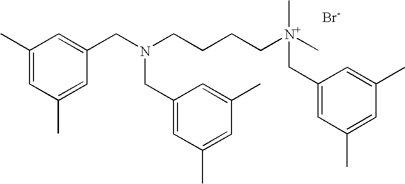 |
| 20 | 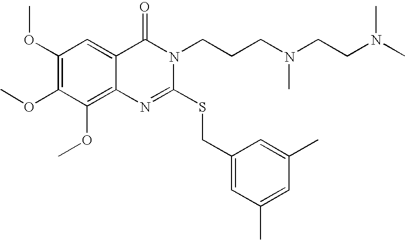 |
| 21 | 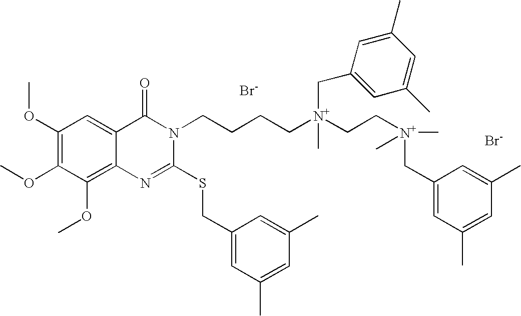 |
| 22 | 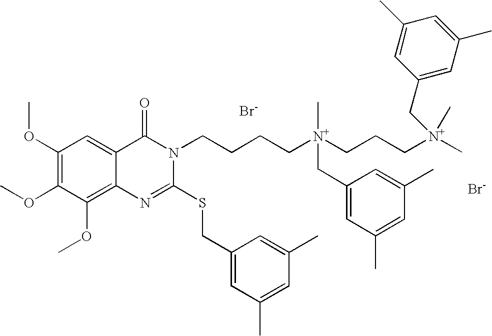 |
| 23 | 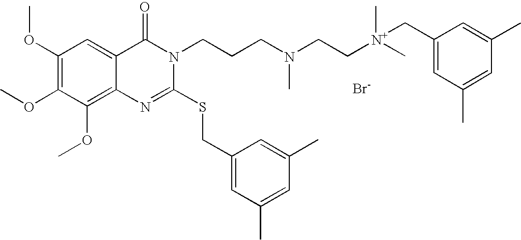 |
| 24 | 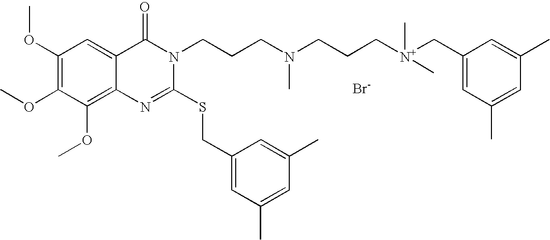 |
| 25 | 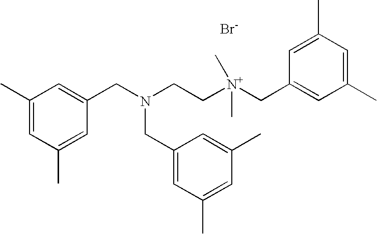 |
| 26 | 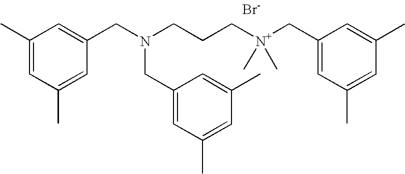 |
| 27 | 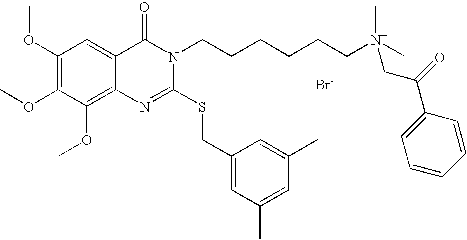 |
| 28 | 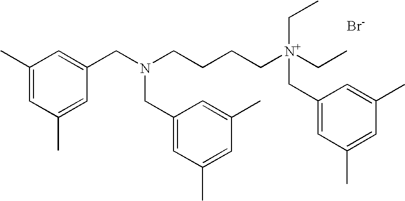 |
| 29 | 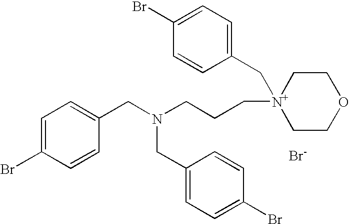 |
| 30 | 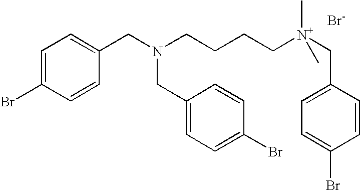 |
| 31 | 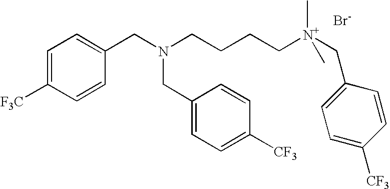 |
| 32 | 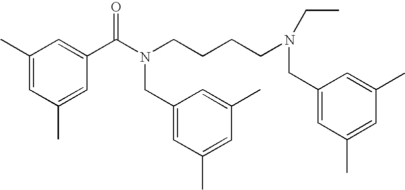 |
| 33 | 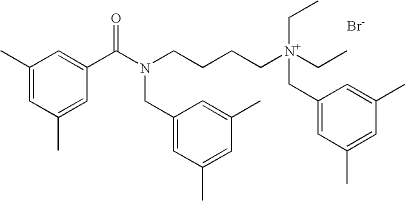 |
| 34 | 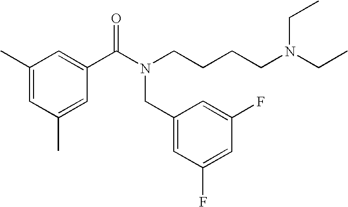 |
| 35 | 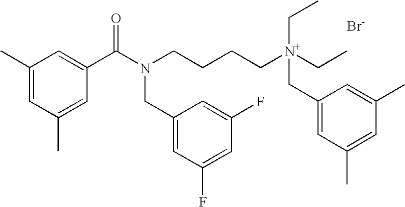 |
| 36 | 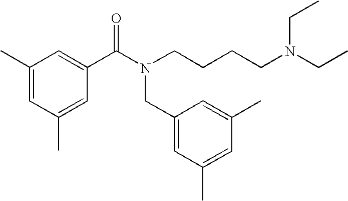 |
| 37 | 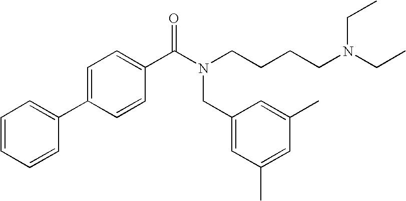 |
| 38 | 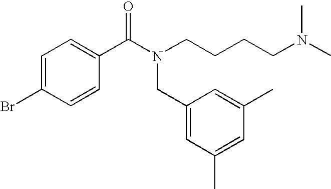 |
| 39 | 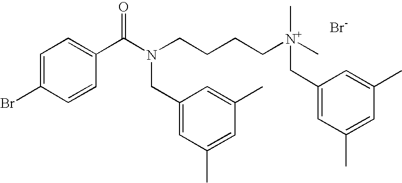 |
| 40 | 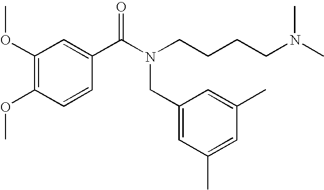 |
| 41 | 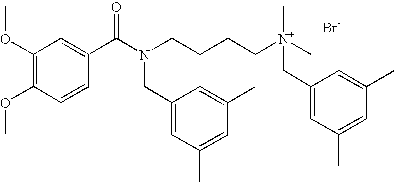 |
| 42 | 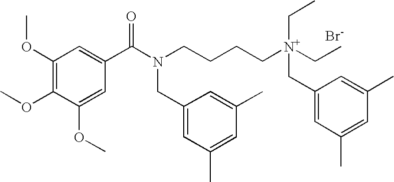 |
| 43 | 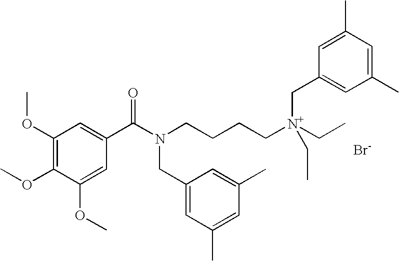 |
| 44 | 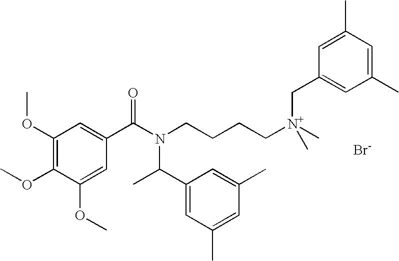 |
| 45 | 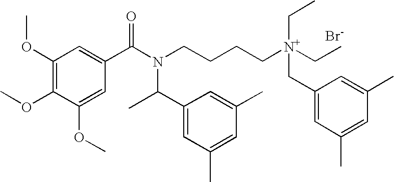 |
| 46 | 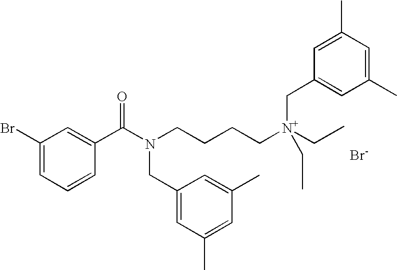 |
| 47 | 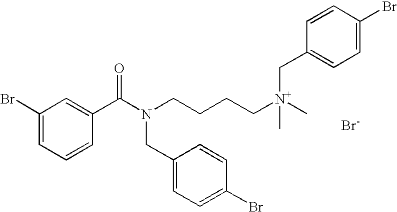 |
| 48 | 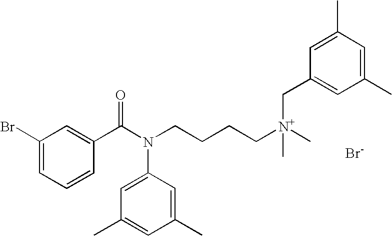 |
| 49 | 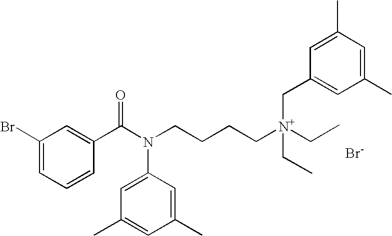 |
| 50 | 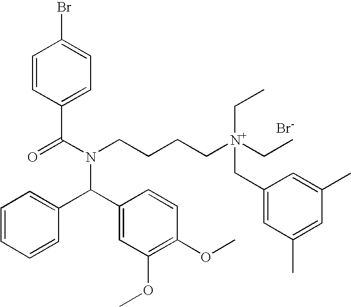 |
| 51 | 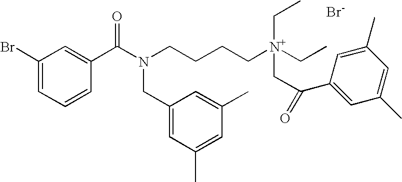 |
| 52 | 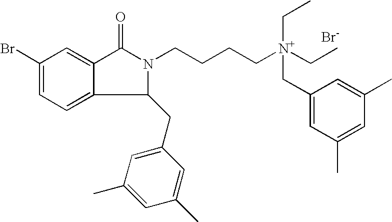 |
| 53 | 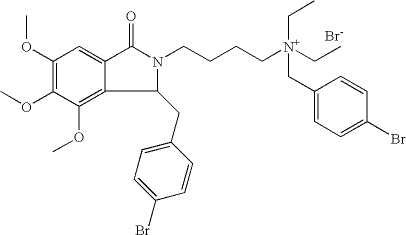 |
| 54 | 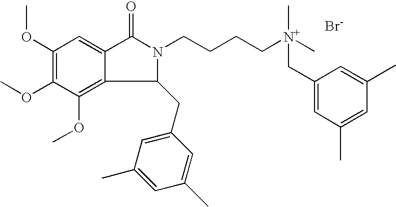 |
| 55 | 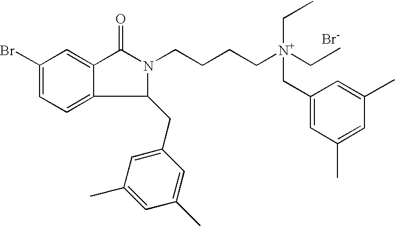 |
| 56 | 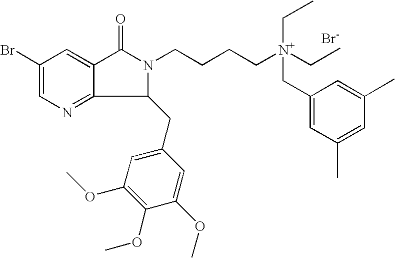 |
| 57 | 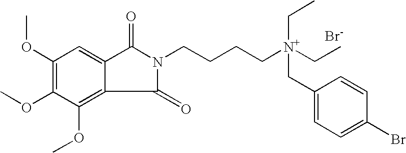 |
| 58 | 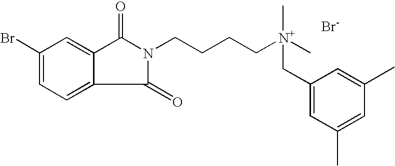 |
| 59 | 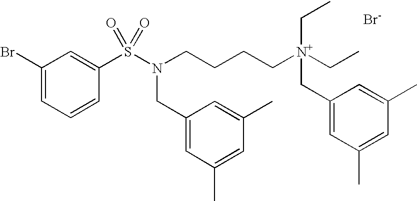 |
| 60 | 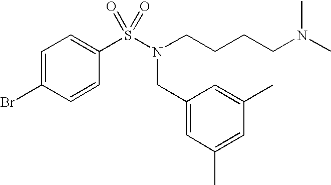 |
| 61 | 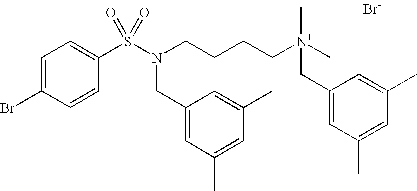 |
| 62 | 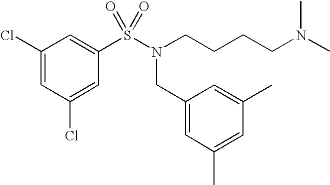 |
| 63 | 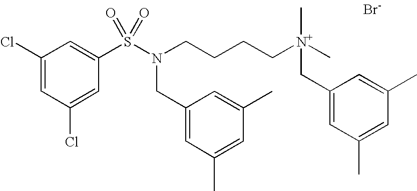 |
| 64 | 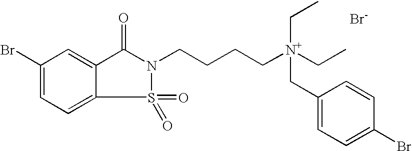 |
| 65 | 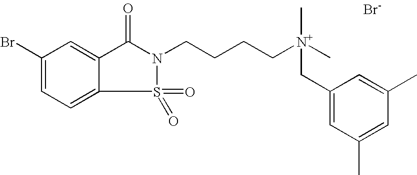 |
| 66 | 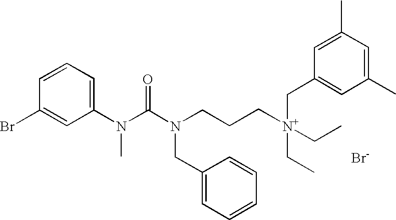 |
| 67 | 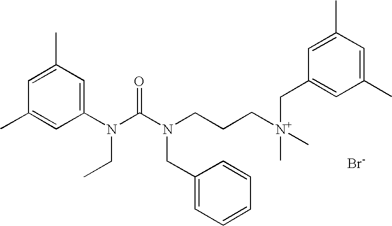 |
| 68 | 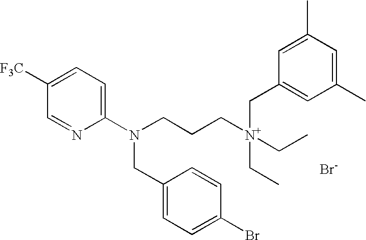 |
| 69 | 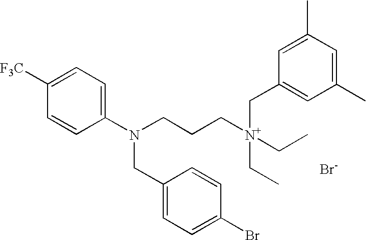 |
| 70 | 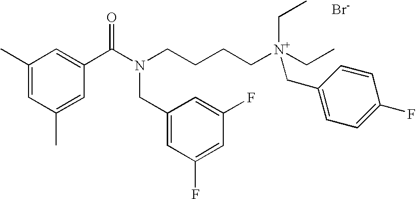 |
| 71 | 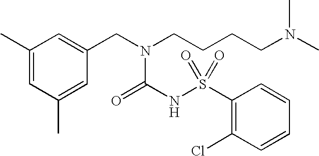 |
| 72 | 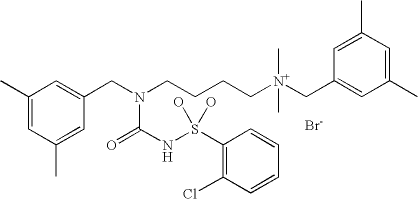 |
| 73 | 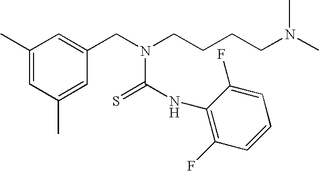 |
| 74 | 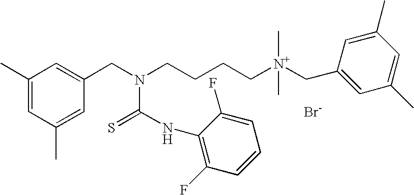 |
| 75 | 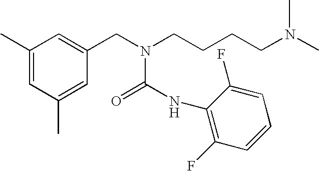 |
| 76 | 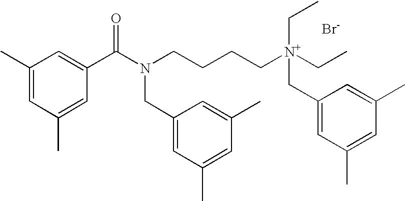 |
| 77 | 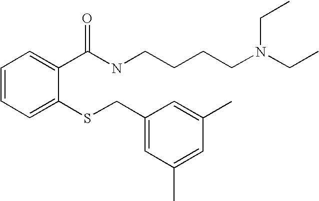 |
| 78 | 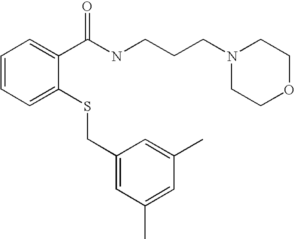 |
| 79 | 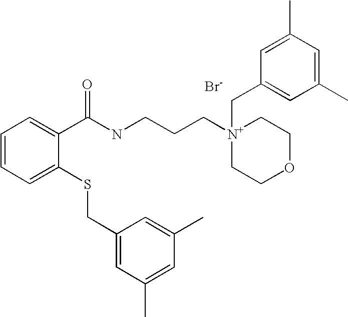 |
| 80 | 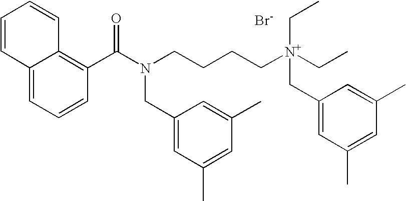 |
| 81 | 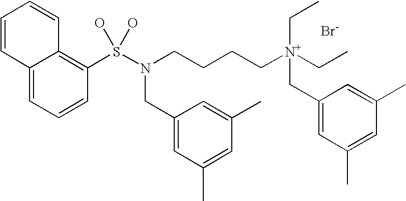 |
| 82 | 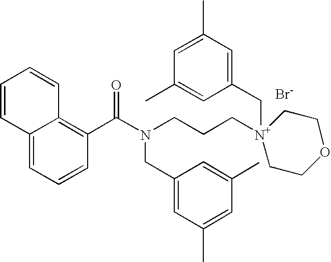 |
| 83 | 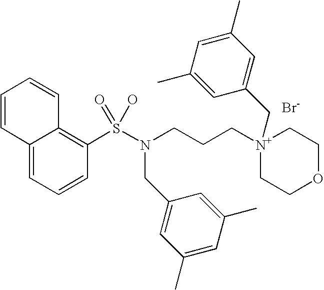 |
| 84 | 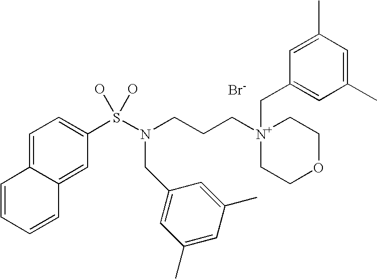 |
| 85 | 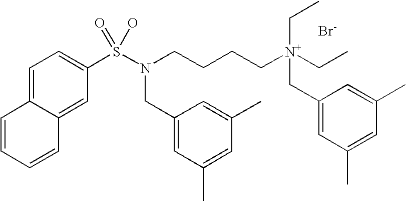 |
| 86 | 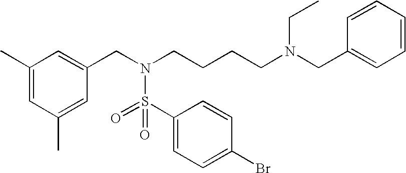 |
| 87 | 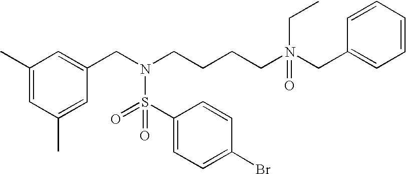 |
| 88 | 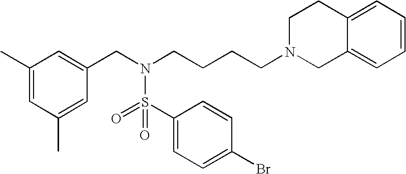 |
| 89 | 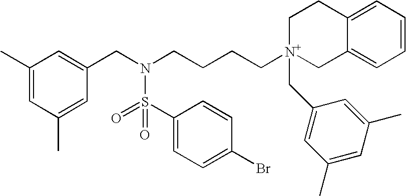 |
| 90 | 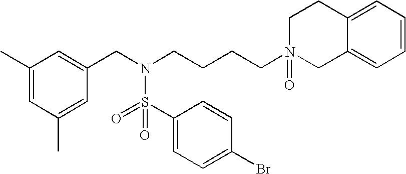 |
| 91 | 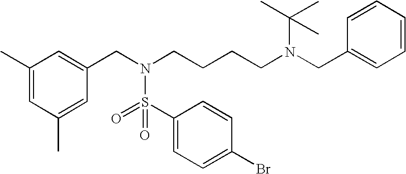 |
| 92 | 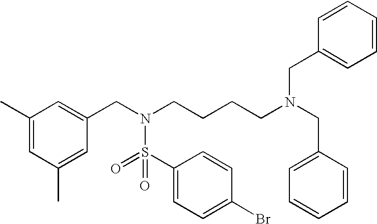 |
| 93 | 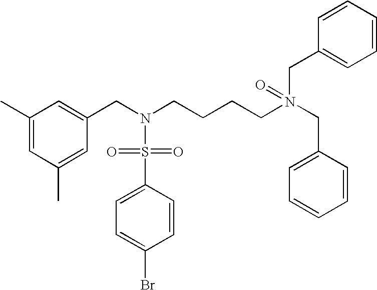 |
| 94 | 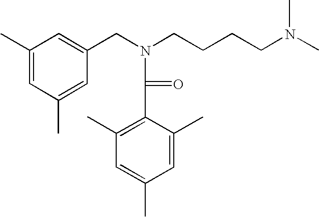 |
| 95 | 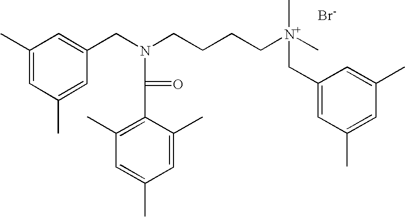 |
| 96 | 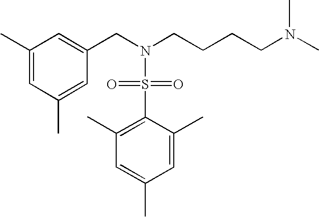 |
| 97 | 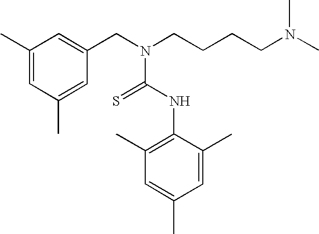 |
| 98 | 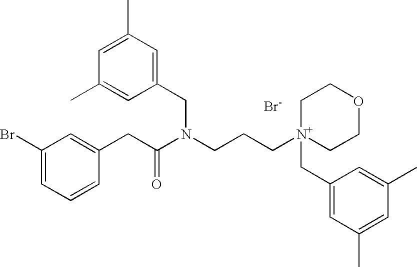 |
| 99 | 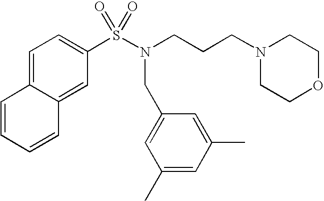 |
| 100 | 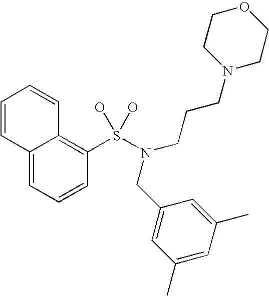 |
| 101 | 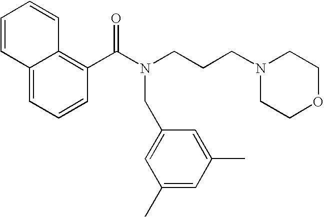 |
| 102 | 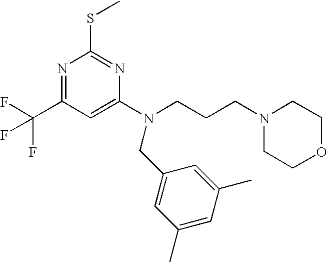 |
| 103 | 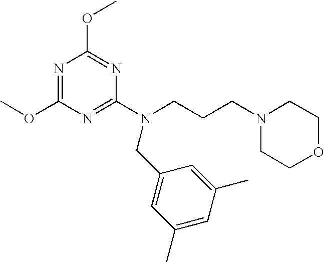 |
| 104 | 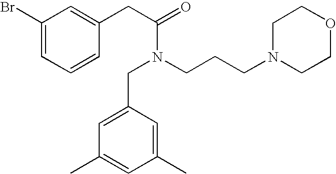 |
| 105 | 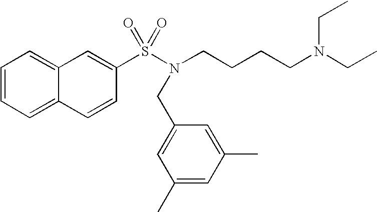 |
| 106 | 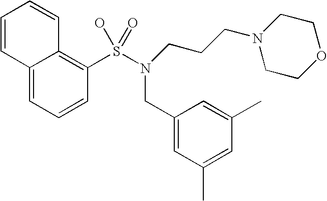 |
| 107 | 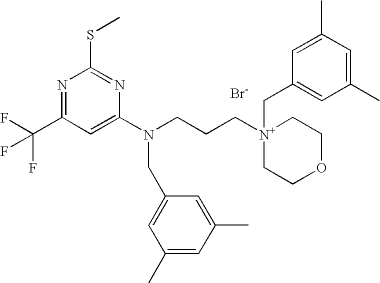 |
| 108 | 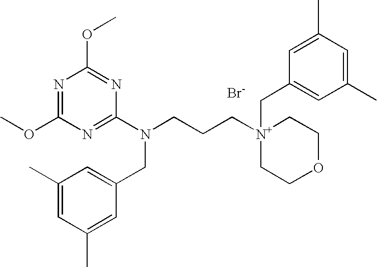 |
| 109 | 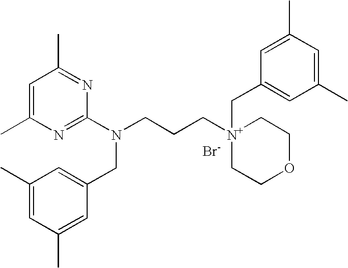 |
| 110 | 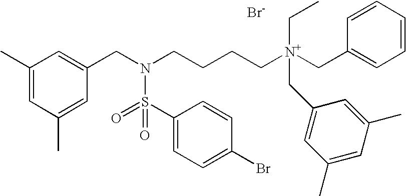 |
| 111 | 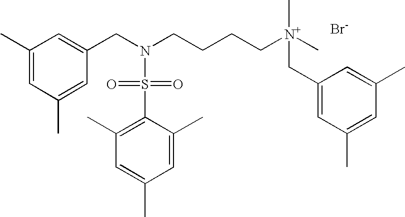 |
| 112 | 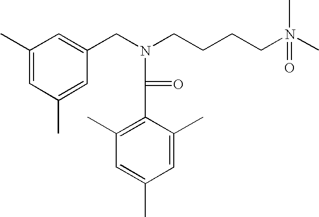 |
| 113 | 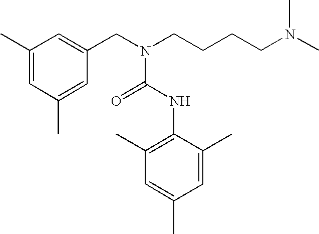 |
| 114 | 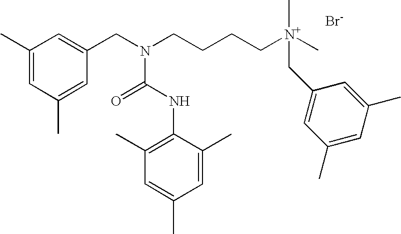 |
| 115 | 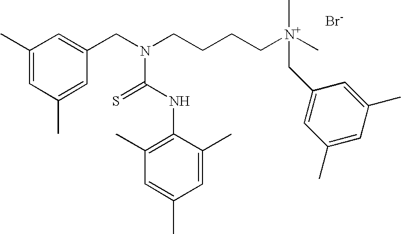 |
| 116 | 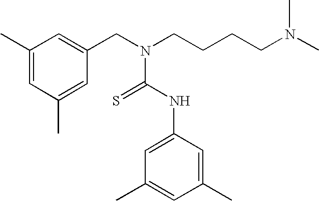 |
| 117 | 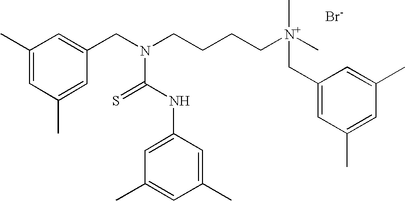 |
| 118 | 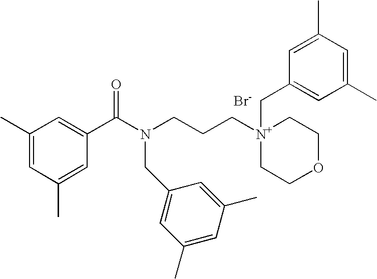 |
| 119 | 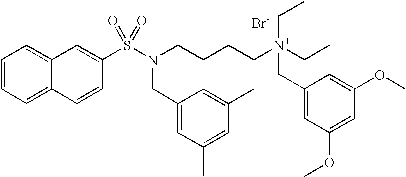 |
| 120 | 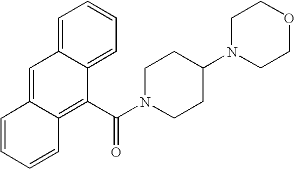 |
| 121 | 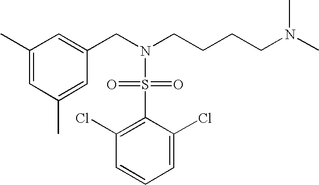 |
| 122 | 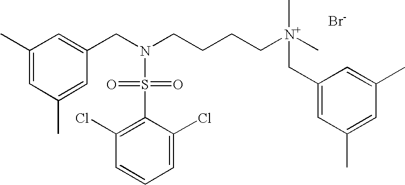 |
| 123 | 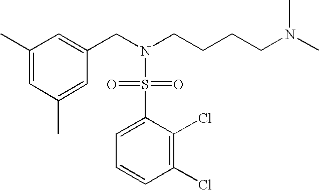 |
| 124 | 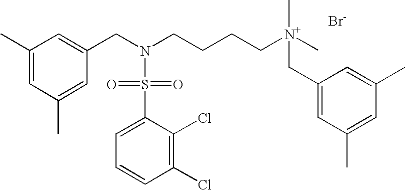 |
| 125 | 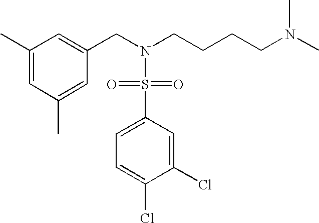 |
| 126 | 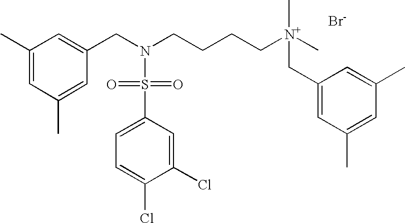 |
| 127 | 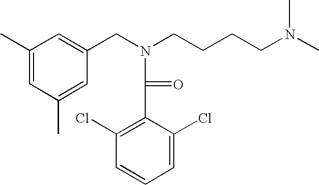 |
| 128 | 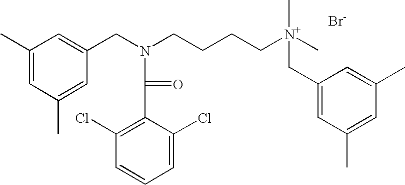 |
| 129 | 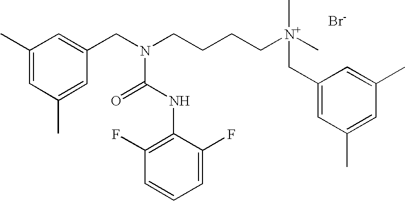 |
| 130 | 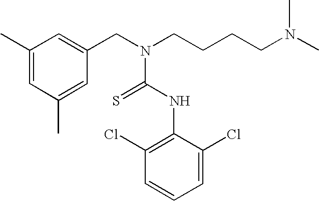 |
| 131 | 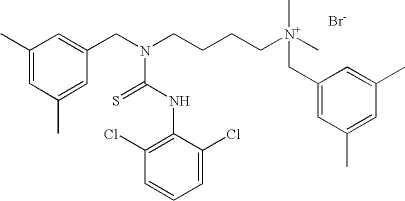 |
| 132 | 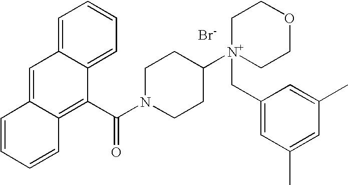 |
| 133 | 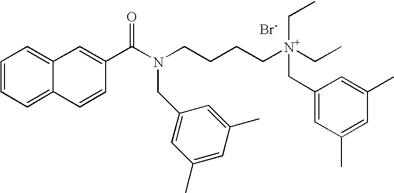 |
| 134 | 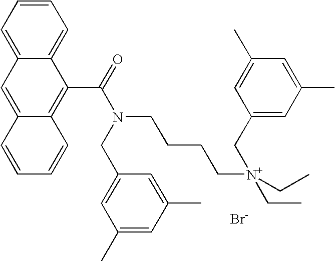 |
| 135 | 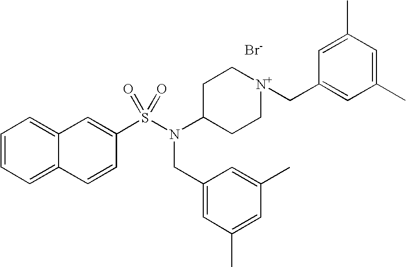 |
| 136 | 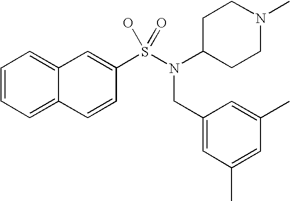 |
| 137 | 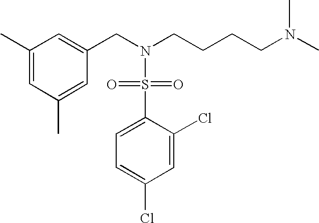 |
| 138 | 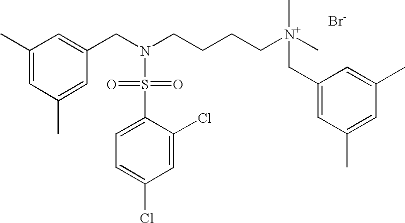 |
| 139 | 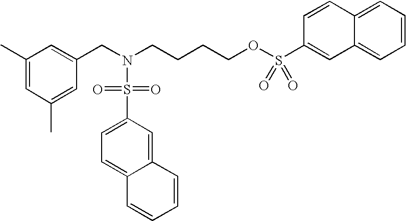 |
| 140 | 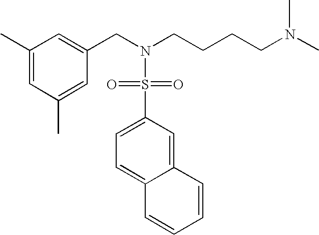 |
| 141 | 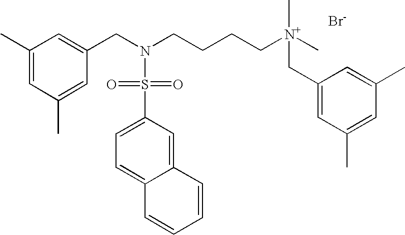 |
| 142 | 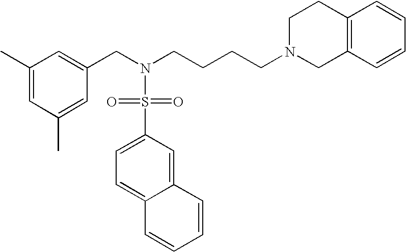 |
| 143 | 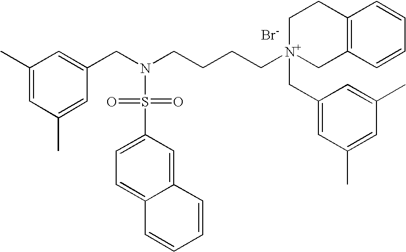 |
| 144 | 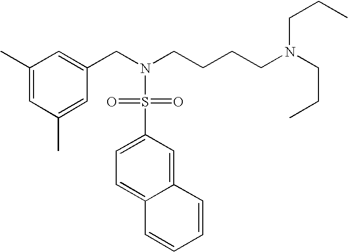 |
| 145 | 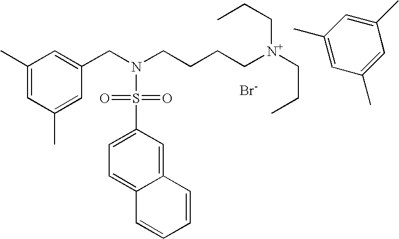 |
| 146 | 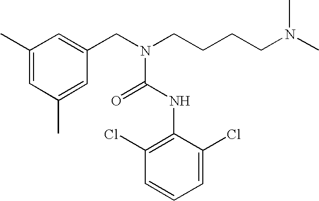 |
| 147 | 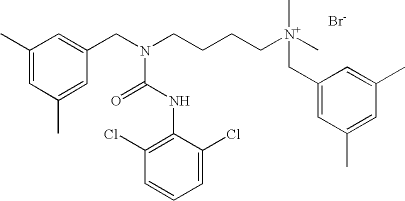 |
| 148 | 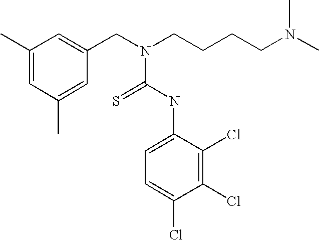 |
| 149 | 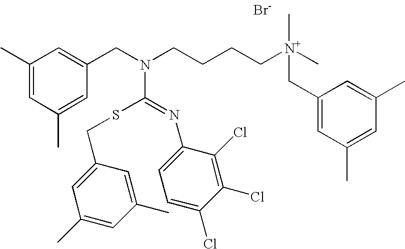 |
| 150 | 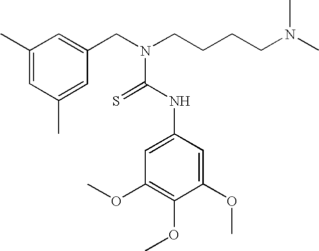 |
| 151 | 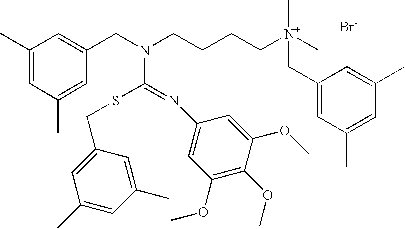 |
| 152 | 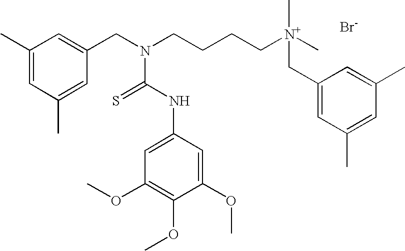 |
| 153 | 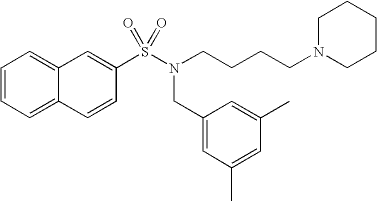 |
| 154 | 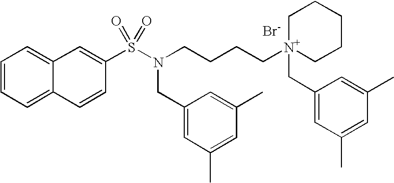 |
| 155 | 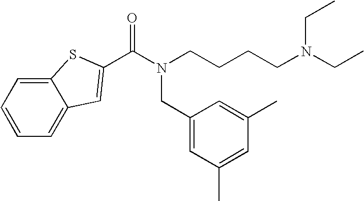 |
| 156 | 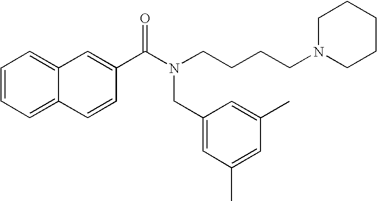 |
| 157 | 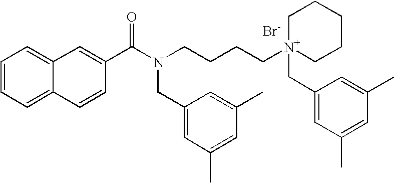 |
| 158 | 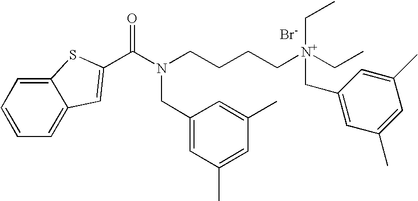 |
| 159 | 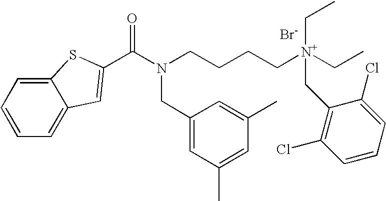 |
| 160 | 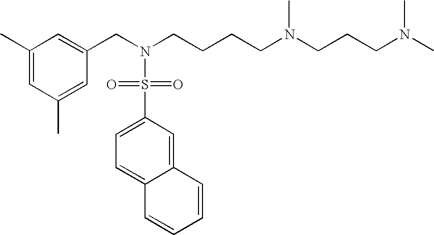 |
| 161 | 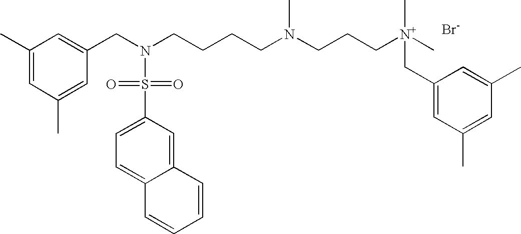 |
| 162 | 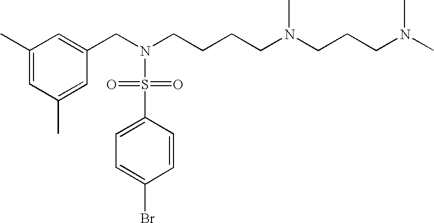 |
| 163 | 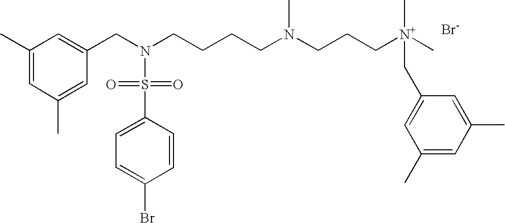 |
| 164 | 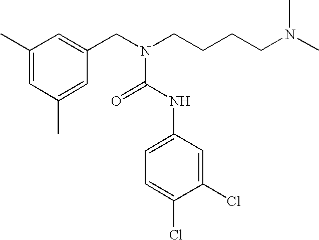 |
| 165 | 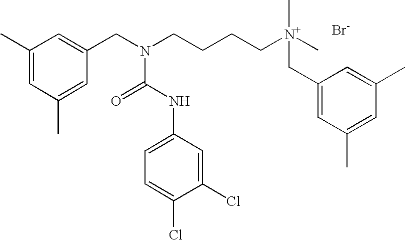 |
| 166 | 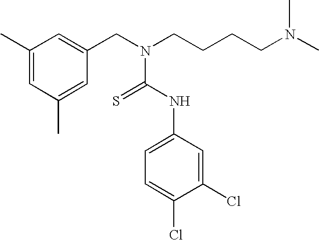 |
| 167 | 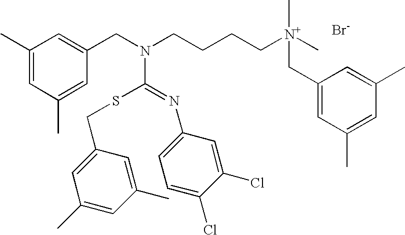 |
| 168 | 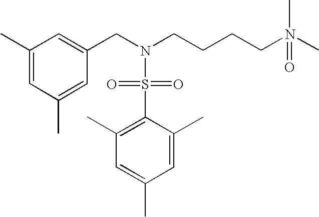 |
| 169 | 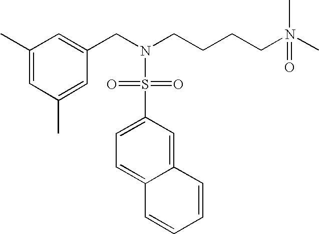 |
| 170 | 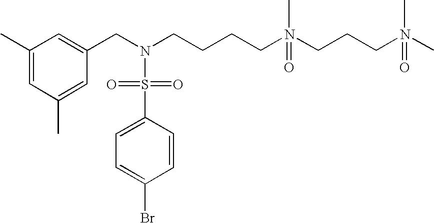 |
| 171 | 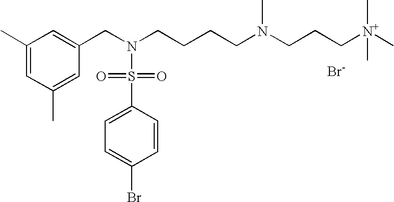 |
| 172 | 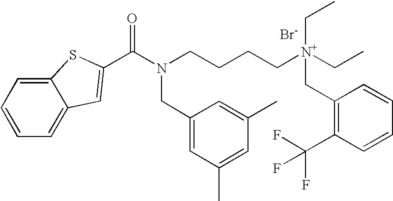 |
| 173 | 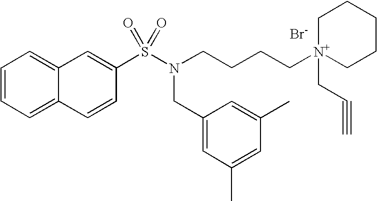 |
| 174 | 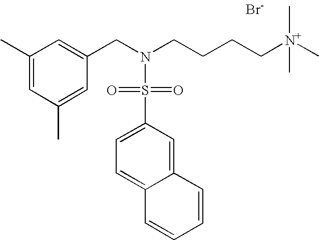 |
| 175 | 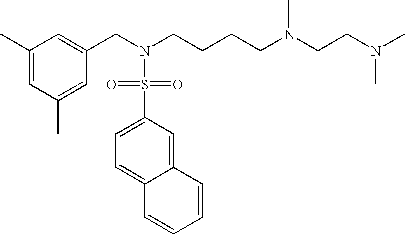 |
| 176 | 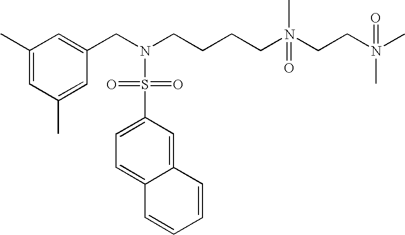 |
| 177 | 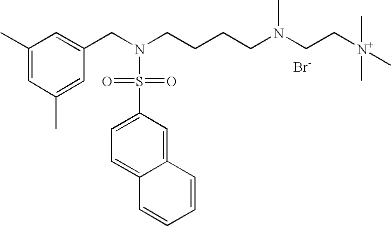 |
| 178 | 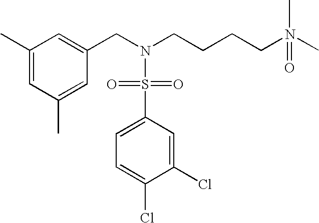 |
| 179 | 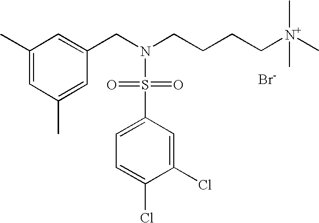 |
| 180 | 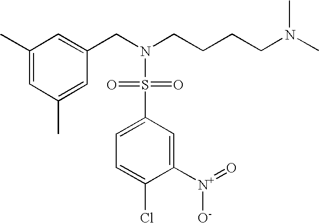 |
| 181 | 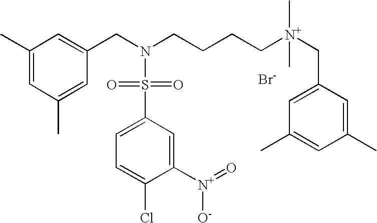 |
| 182 | 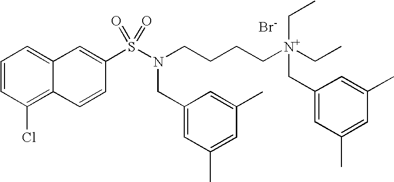 |
| 183 | 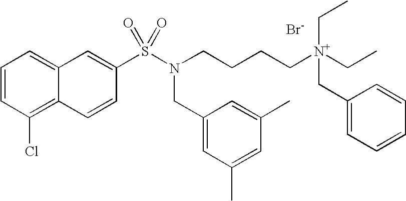 |
| 184 | 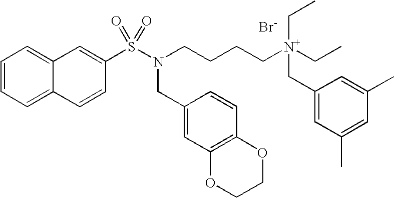 |
| 185 | 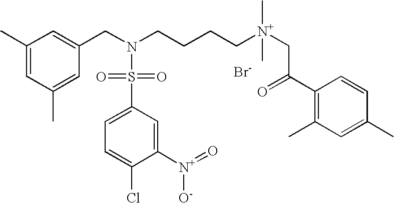 |
| 186 | 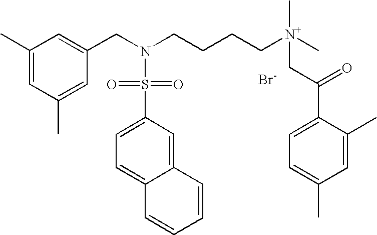 |
| 187 | 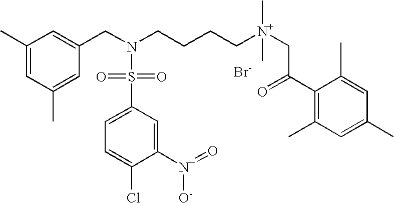 |
| 188 | 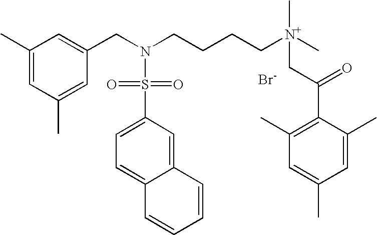 |
| 189 | 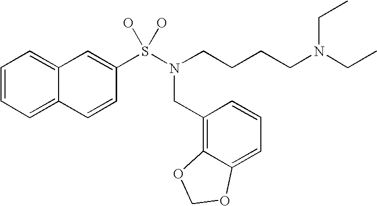 |
| 190 | 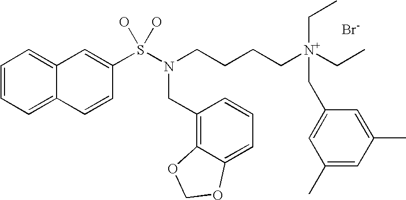 |
| 191 | 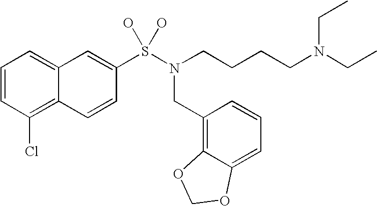 |
| 192 | 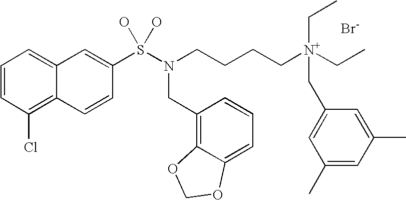 |
| 193 | 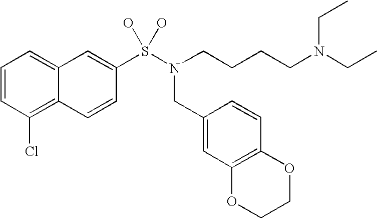 |
| 194 | 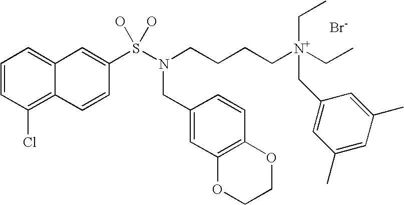 |
| 195 | 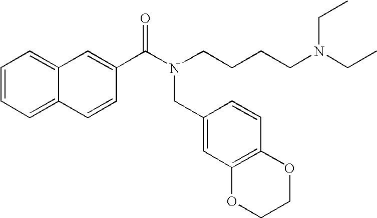 |
| 196 | 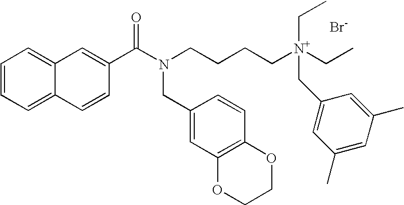 |
| 197 | 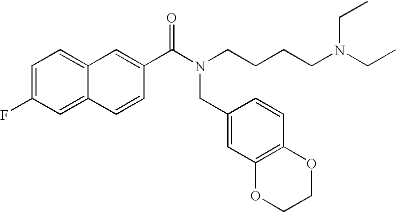 |
| 198 | 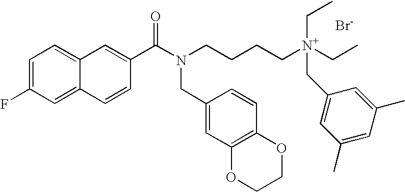 |
| 199 | 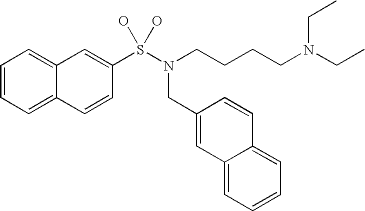 |
| 200 | 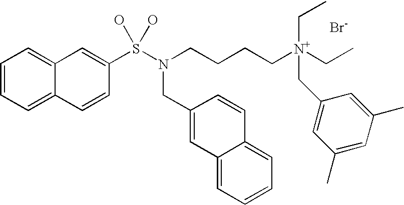 |
| 201 | 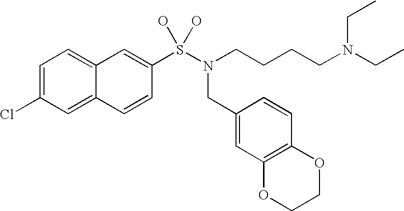 |
| 202 | 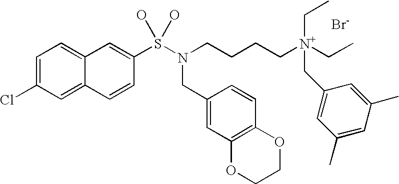 |
| 203 | 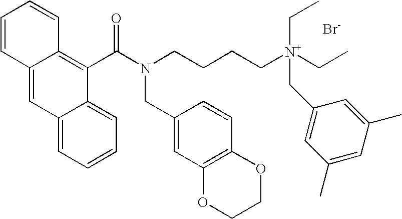 |
| 204 | 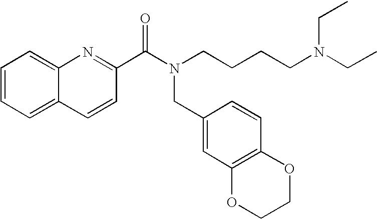 |
| 205 | 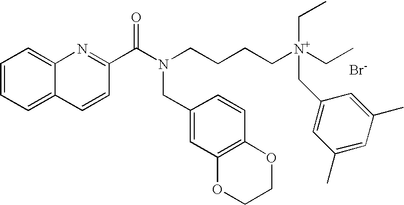 |
| 206 | 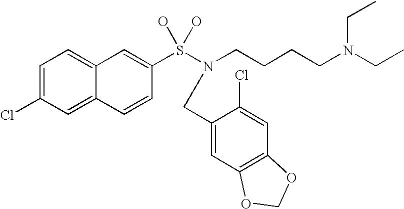 |
| 207 | 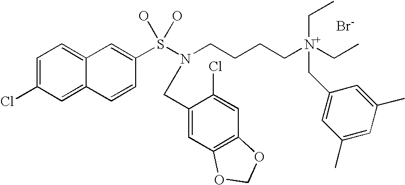 |
| 208 | 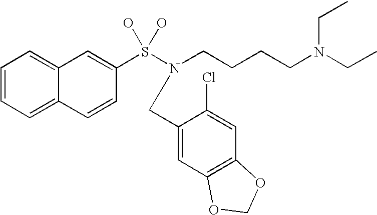 |
| 209 | 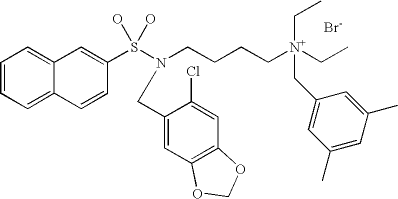 |
| 210 | 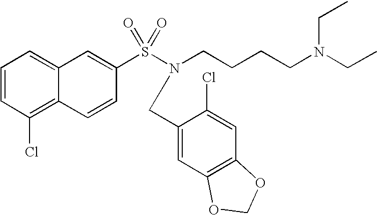 |
| 211 | 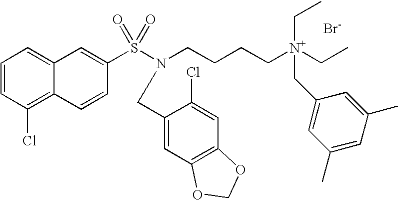 |
| 212 | 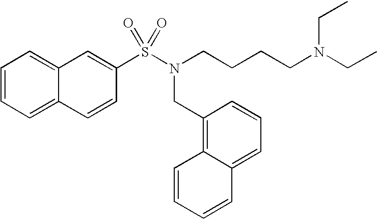 |
| 213 | 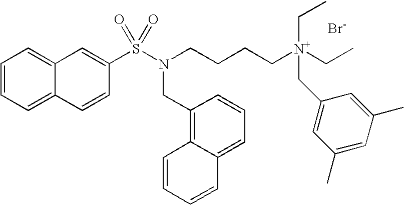 |
| 214 | 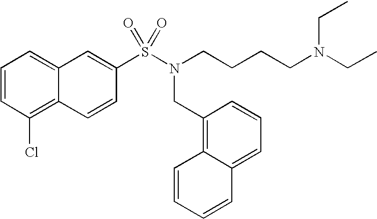 |
| 215 | 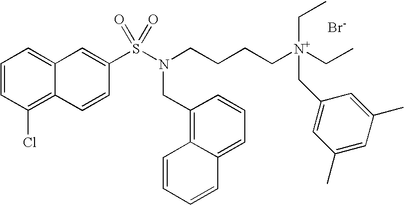 |
| 216 | 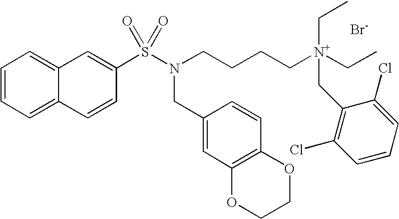 |
| 217 | 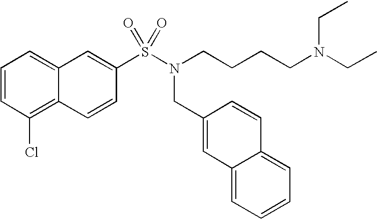 |
| 218 | 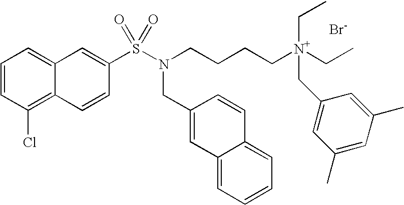 |
| 219 | 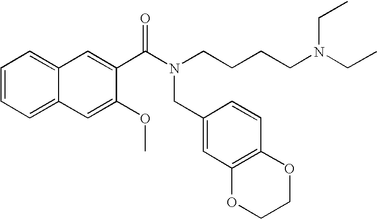 |
| 220 | 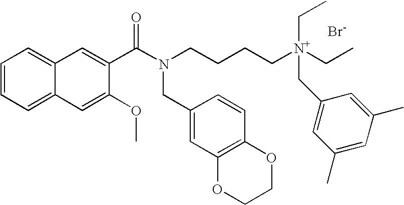 |
| 221 | 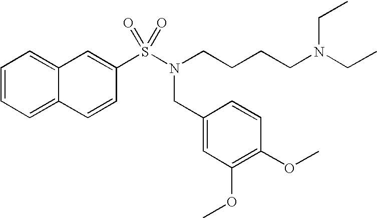 |
| 222 | 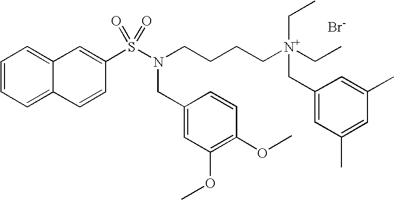 |
| 223 | 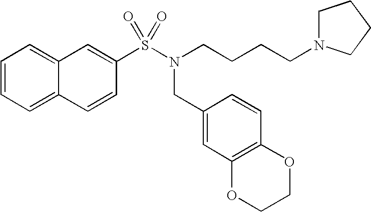 |
| 224 | 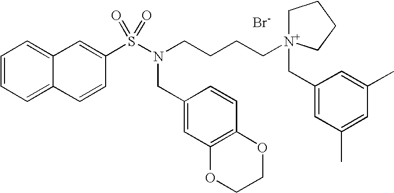 |
| 225 | 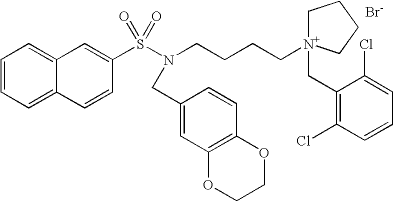 |
| 226 | 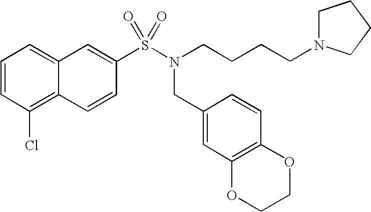 |
| 227 | 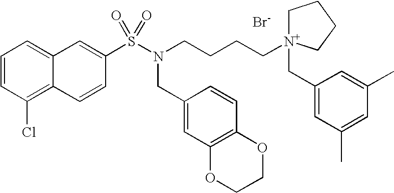 |
| 228 | 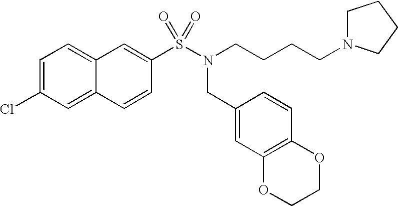 |
| 229 | 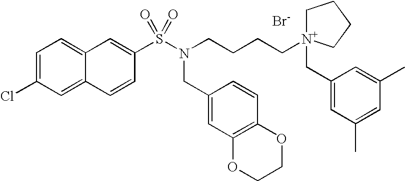 |
| 230 | 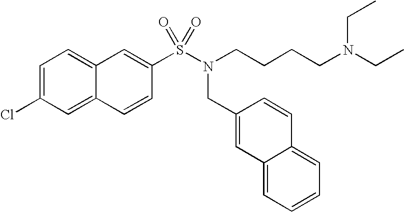 |
| 231 | 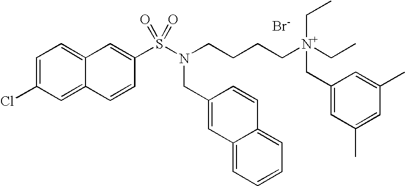 |
| 232 | 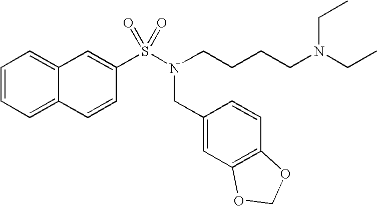 |
| 233 | 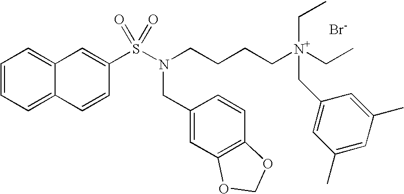 |
| 234 | 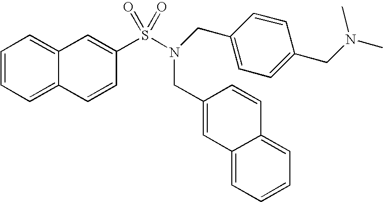 |
| 235 | 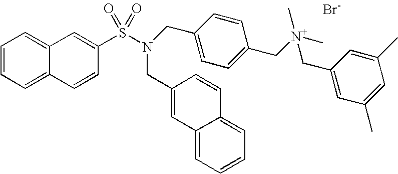 |
| 236 | 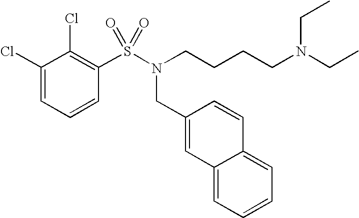 |
| 237 | 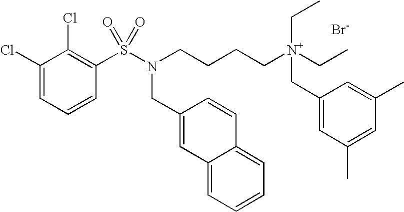 |
| 238 | 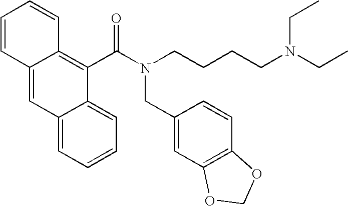 |
| 239 | 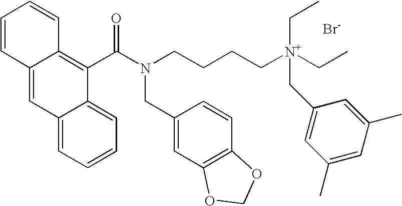 |
| 240 | 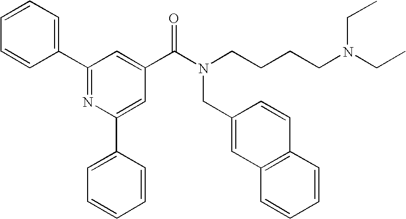 |
| 241 | 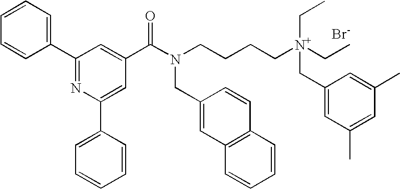 |
| 242 | 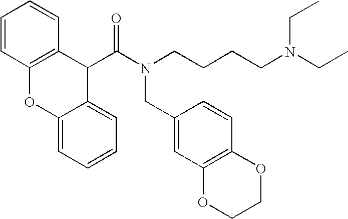 |
| 243 | 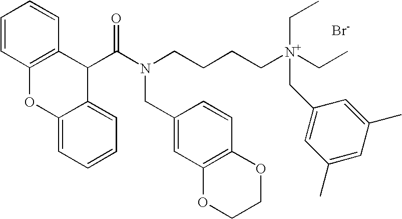 |
| 244 | 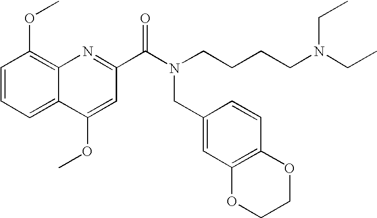 |
| 245 | 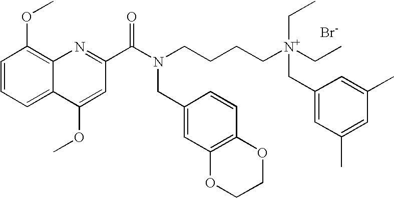 |
| 246 | 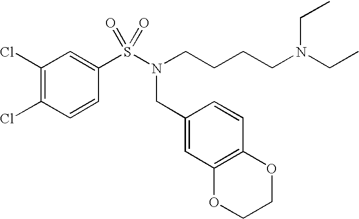 |
| 247 | 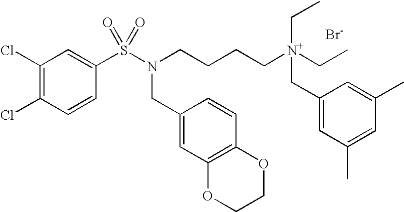 |
| 248 | 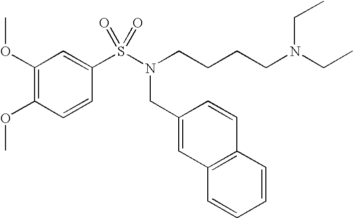 |
| 249 | 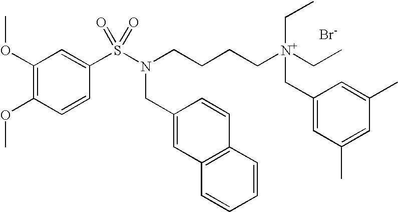 |
| 250 | 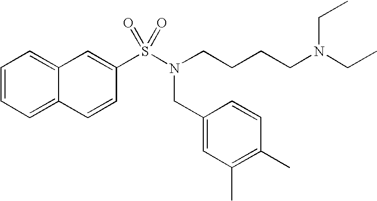 |
| 251 | 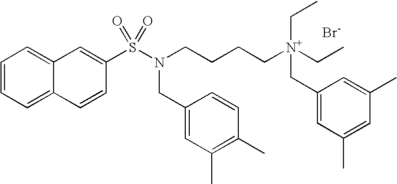 |
| 252 | 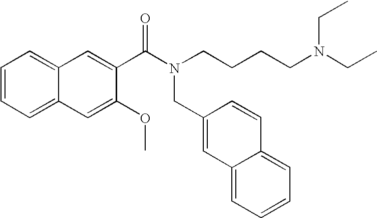 |
| 253 | 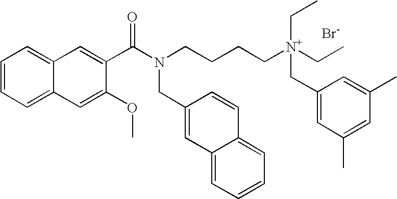 |
| 254 | 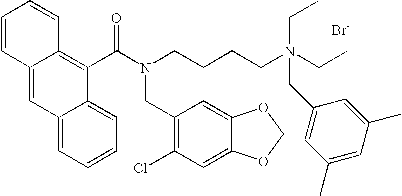 |
| 255 | 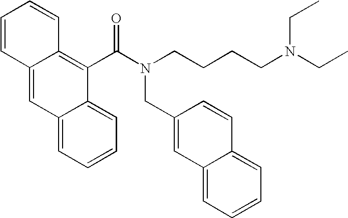 |
| 256 | 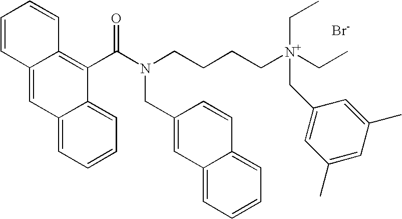 |
| 257 | 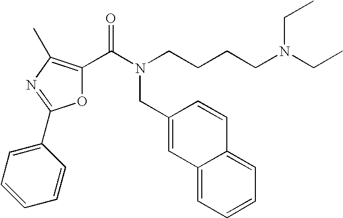 |
| 258 | 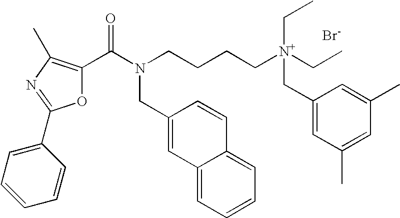 |
| 259 | 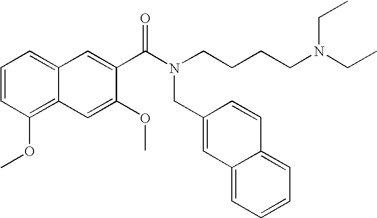 |
| 260 | 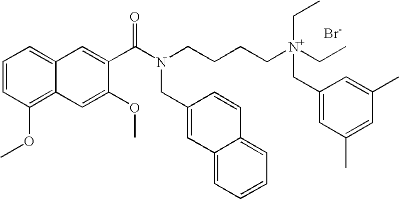 |
| 261 | 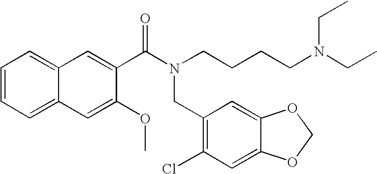 |
| 262 | 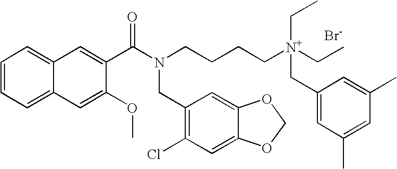 |
| 263 | 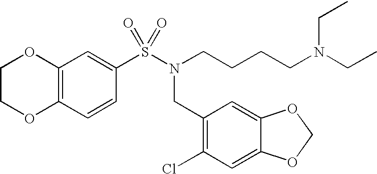 |
| 264 | 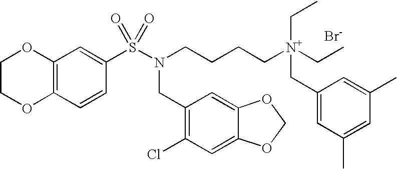 |
| 265 | 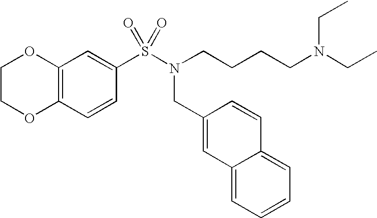 |
| 266 | 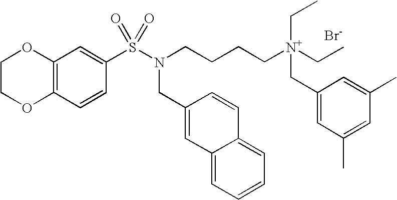 |
| 267 | 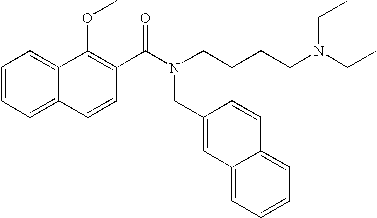 |
| 268 | 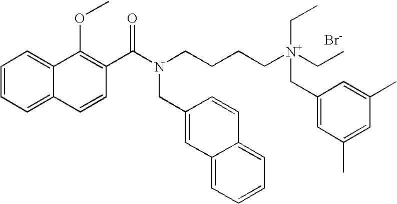 |
| 269 | 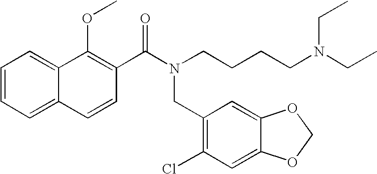 |
| 270 | 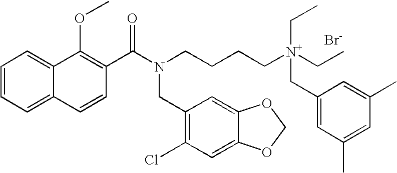 |
| 271 | 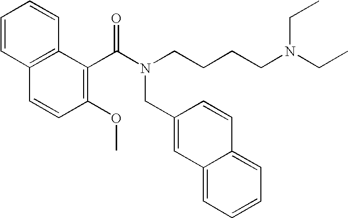 |
| 272 | 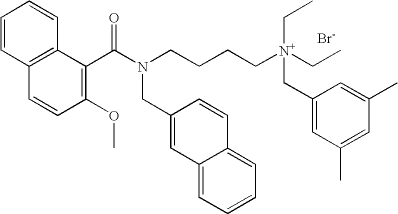 |
| 273 | 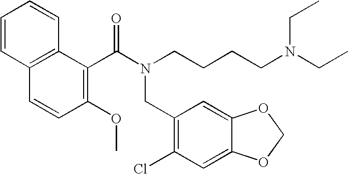 |
| 274 | 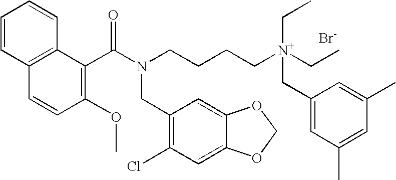 |
| 275 | 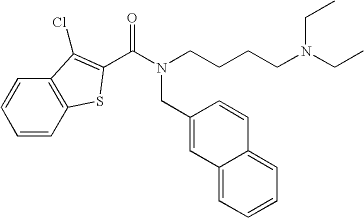 |
| 276 | 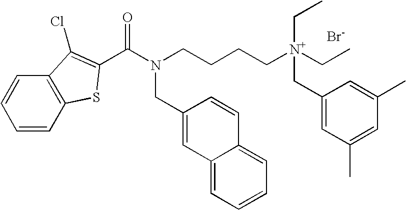 |
| 277 | 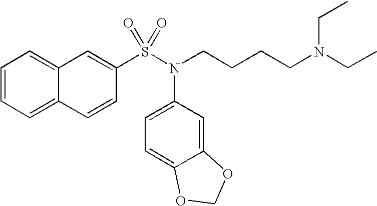 |
| 278 | 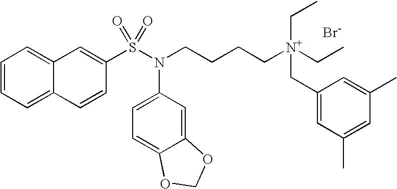 |
| 279 | 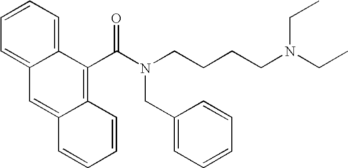 |
| 280 | 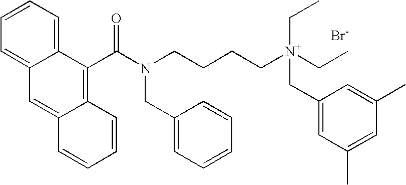 |
| 281 | 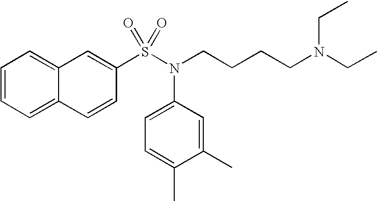 |
| 282 | 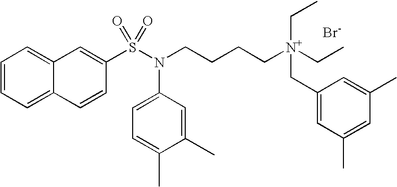 |
| 283 | 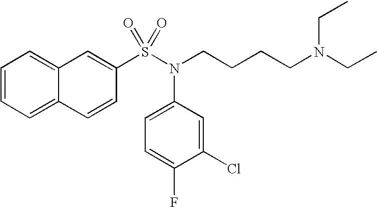 |
| 284 | 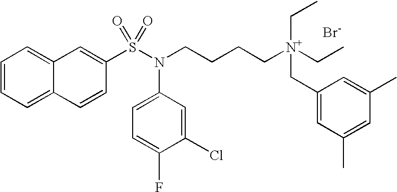 |
| 285 | 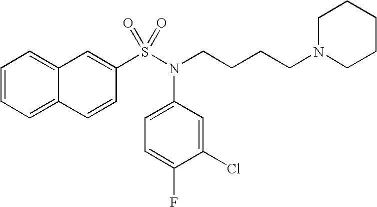 |
| 286 | 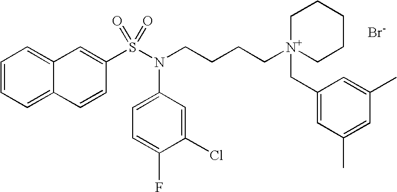 |
| 287 | 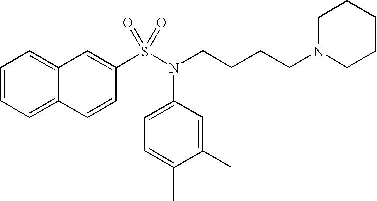 |
| 288 | 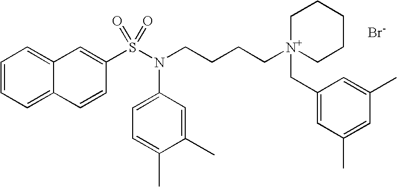 |
| 289 | 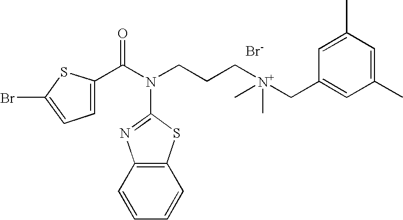 |
| 290 | 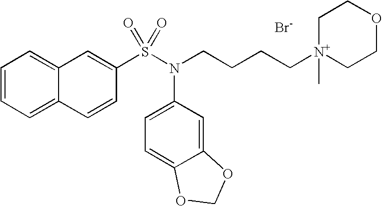 |
| 291 | 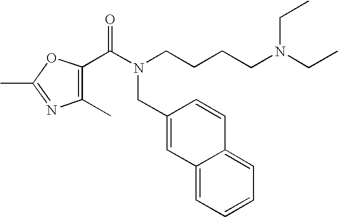 |
| 292 | 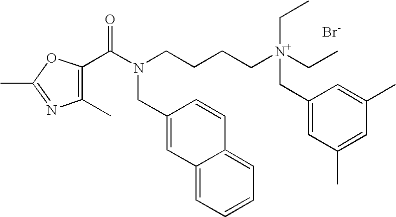 |
| 293 | 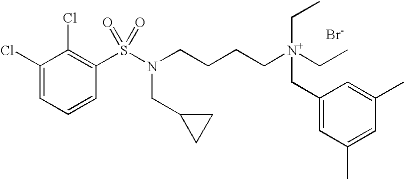 |
| 294 | 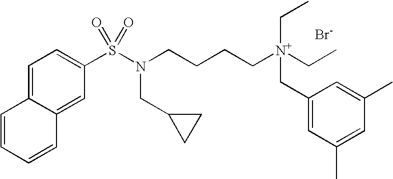 |
| 295 | 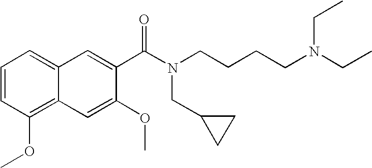 |
| 296 | 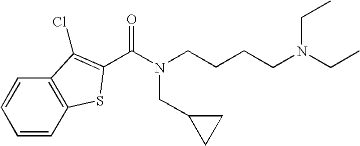 |
| 297 | 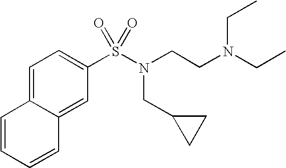 |
| 298 | 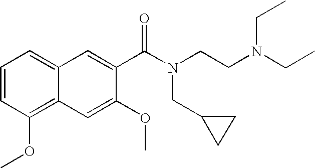 |
| 299 | 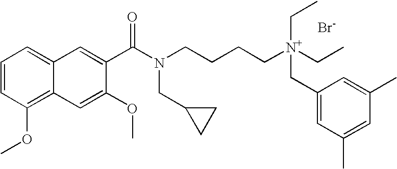 |
| 300 | 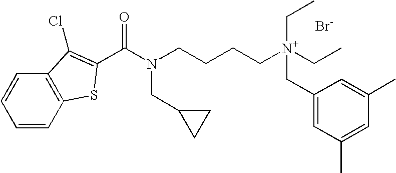 |
| 301 | 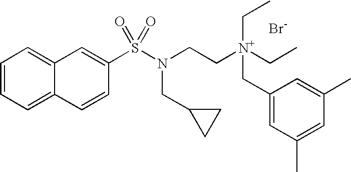 |
| 302 | 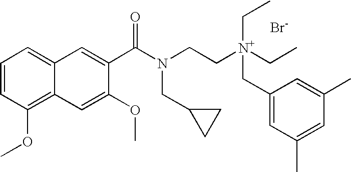 |
| 303 | 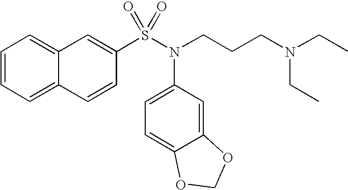 |
| 304 | 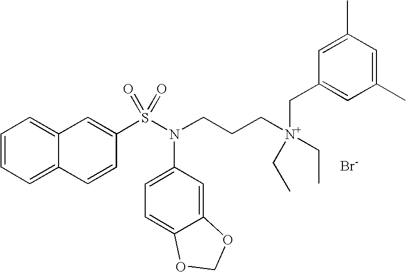 |
| 305 | 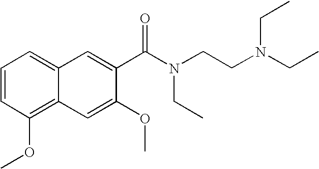 |
| 306 | 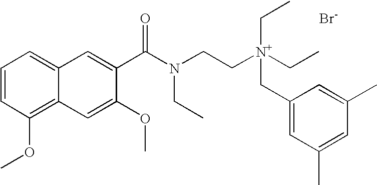 |
| 307 | 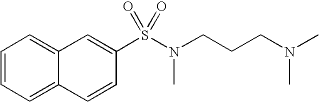 |
| 308 | 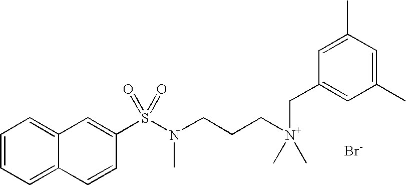 |
| 309 | 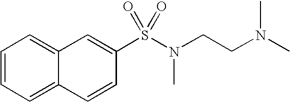 |
| 310 | 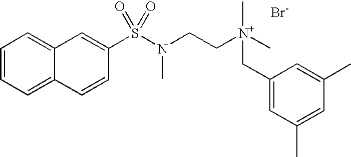 |
| 311 | 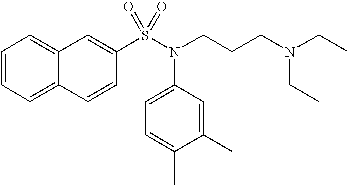 |
| 312 | 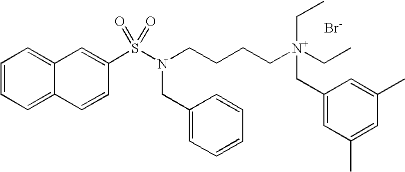 |
| 313 | 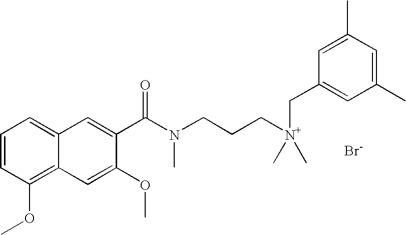 |
| 314 | 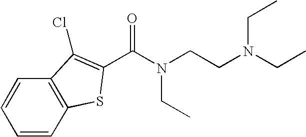 |
| 315 | 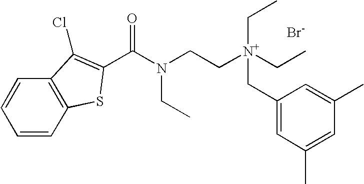 |
| 316 | 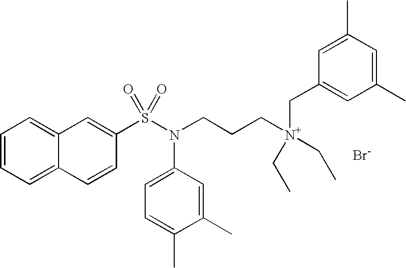 |
| 317 | 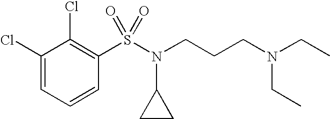 |
| 318 | 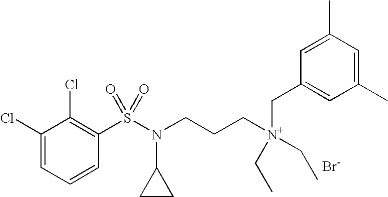 |
| 319 | 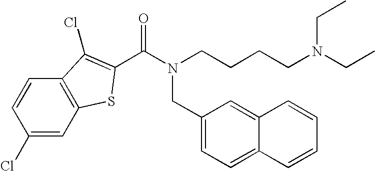 |
| 320 | 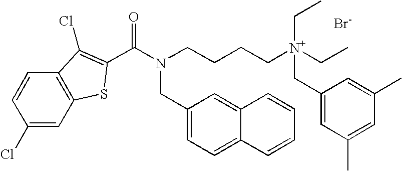 |
| 321 | 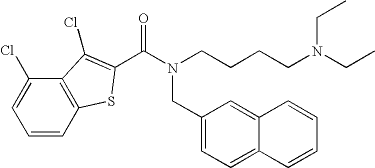 |
| 322 | 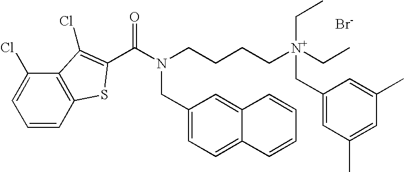 |
| 323 | 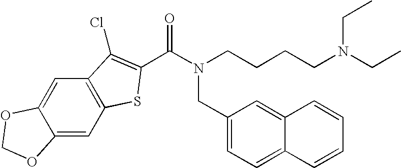 |
| 324 | 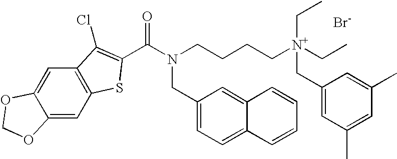 |
| 325 | 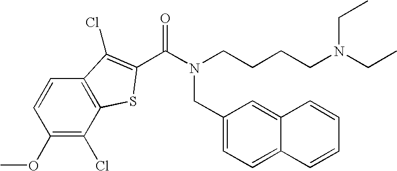 |
| 326 | 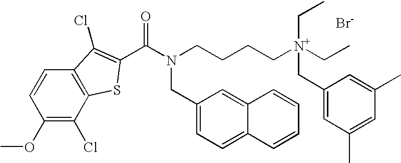 |
| 327 | 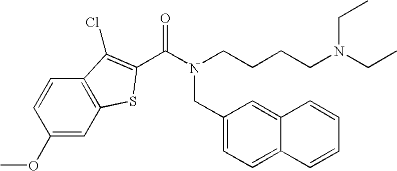 |
| 328 | 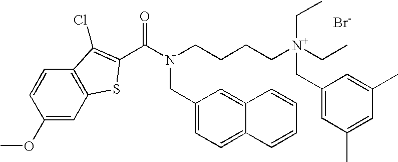 |
| 329 | 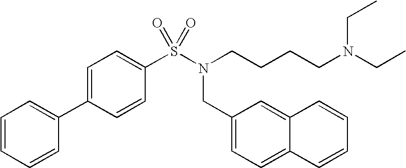 |
| 330 | 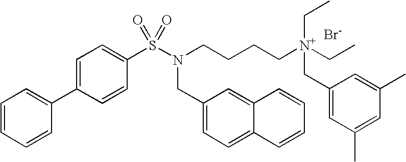 |
| 331 | 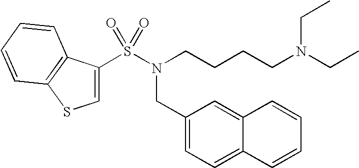 |
| 332 | 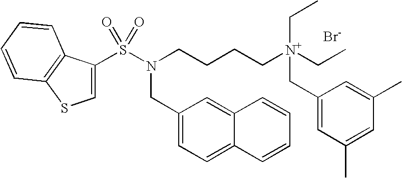 |
| 333 | 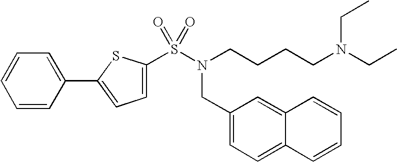 |
| 334 | 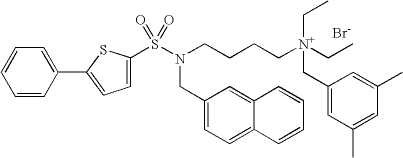 |
| 335 | 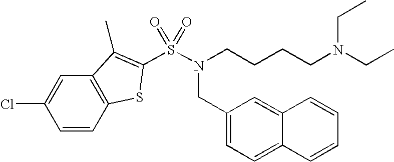 |
| 336 | 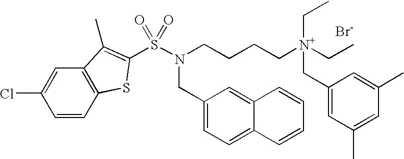 |
| 337 | 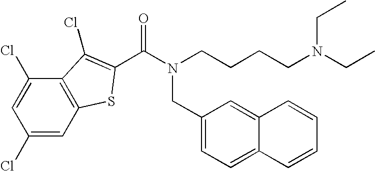 |
| 338 | 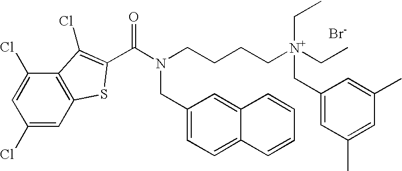 |
| 339 | 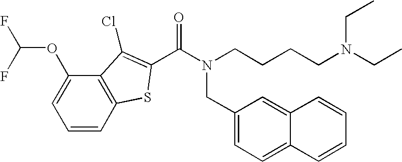 |
| 340 | 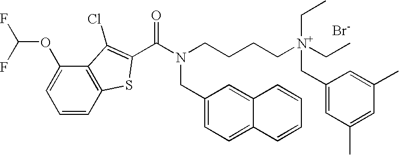 |
| 341 | 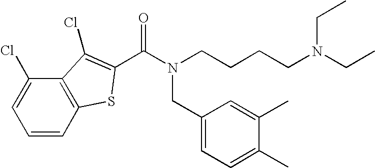 |
| 342 | 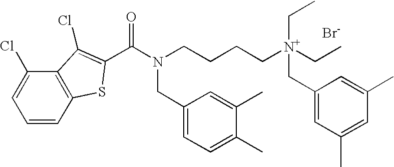 |
| 343 | 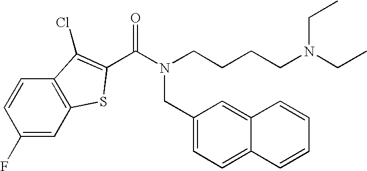 |
| 344 | 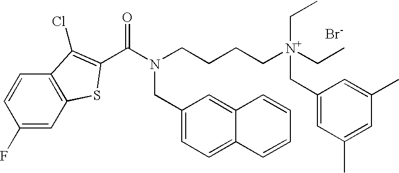 |
| 345 | 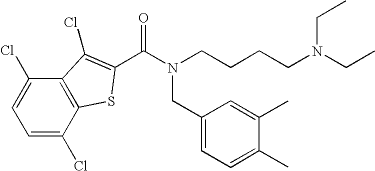 |
| 346 | 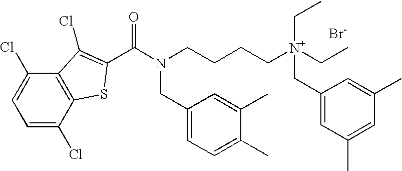 |
| 347 | 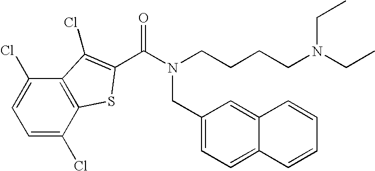 |
| 348 | 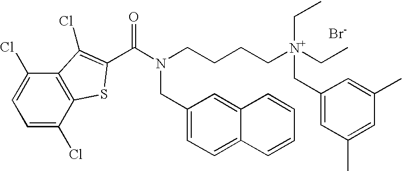 |
| 349 | 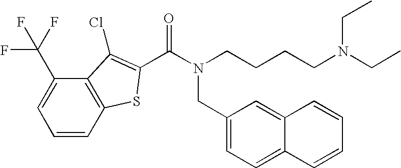 |
| 350 | 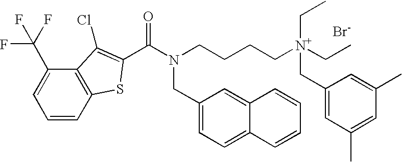 |
| 351 | 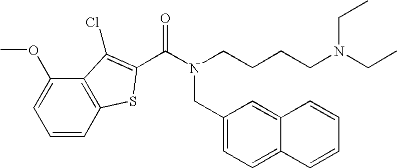 |
| 352 | 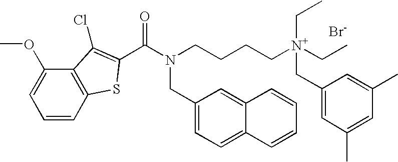 |
| 353 | 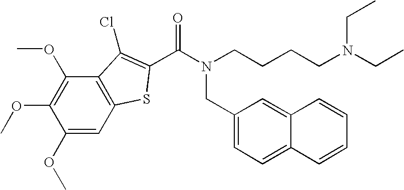 |
| 354 | 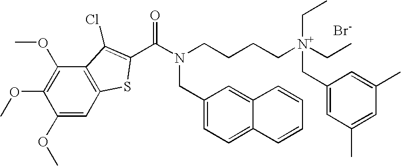 |
| 355 | 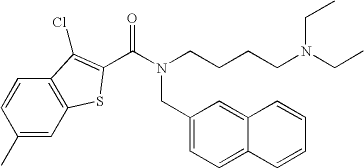 |
| 356 | 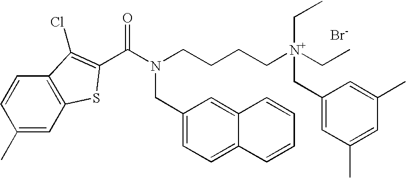 |
| 357 | 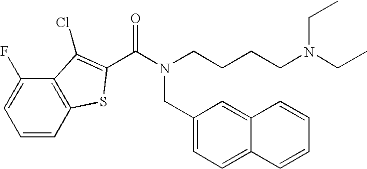 |
| 358 | 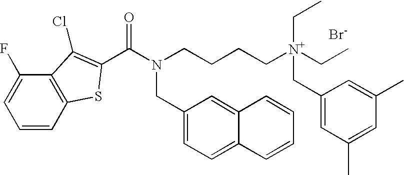 |
| 359 | 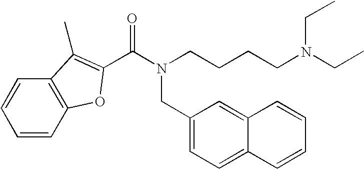 |
| 360 | 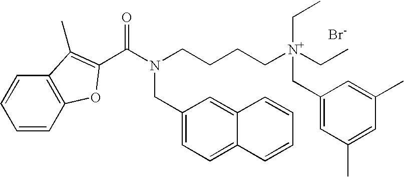 |
| 361 | 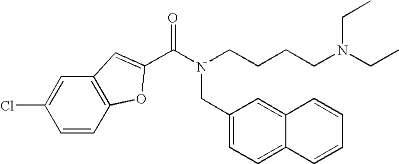 |
| 362 | 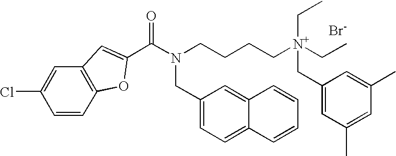 |
| 363 | 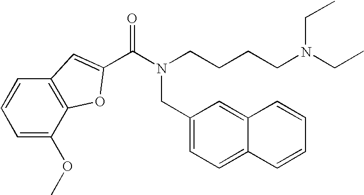 |
| 364 | 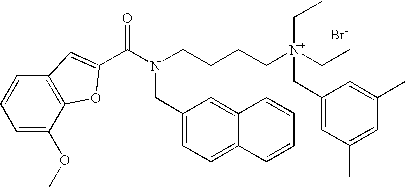 |
| 365 | 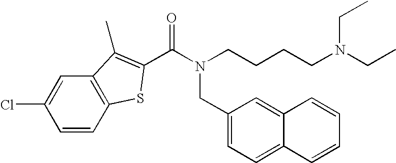 |
| 366 | 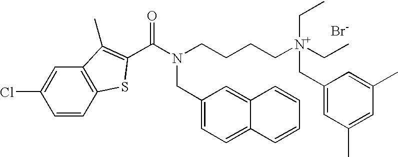 |
| 367 | 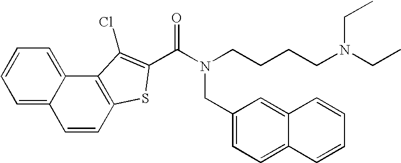 |
| 368 | 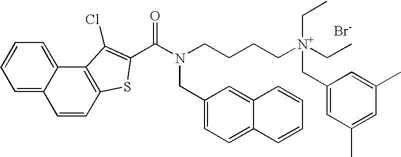 |
| 369 | 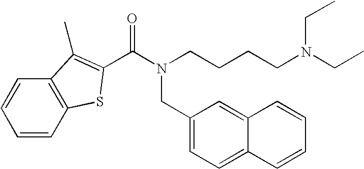 |
| 370 | 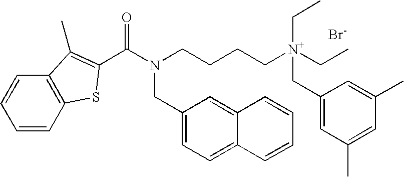 |
| 371 | 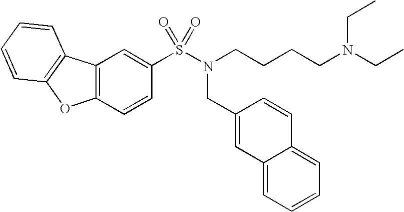 |
| 372 | 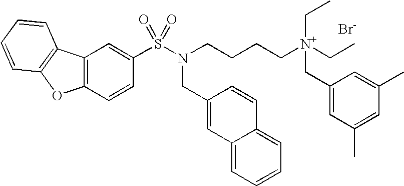 |
| 373 | 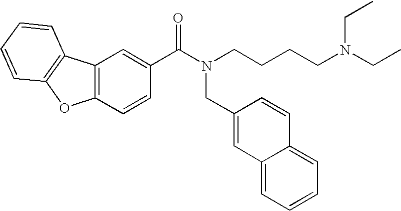 |
| 374 | 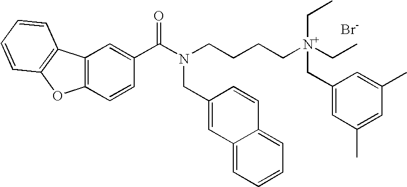 |
| 375 | 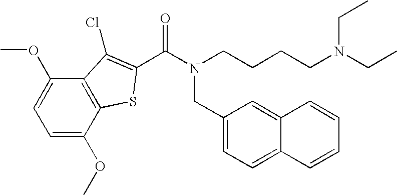 |
| 376 | 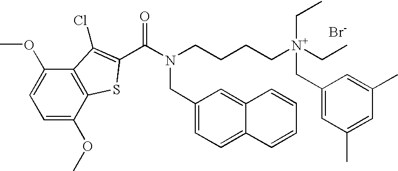 |
| 377 | 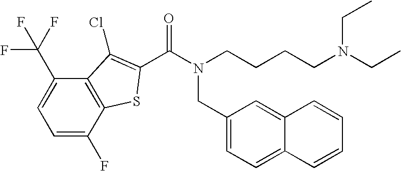 |
| 378 | 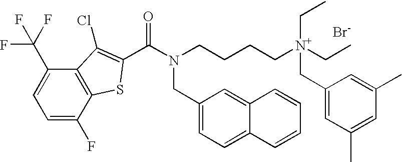 |
| 379 | 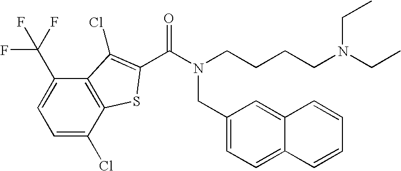 |
| 380 | 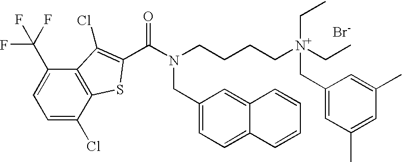 |
| 381 | 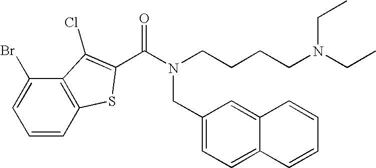 |
| 382 | 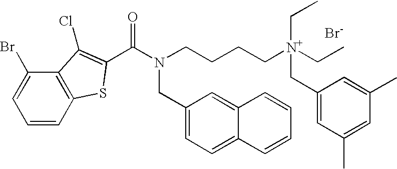 |
| 383 | 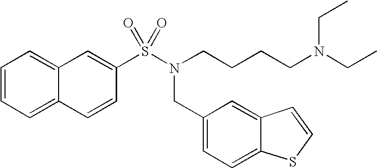 |
| 384 | 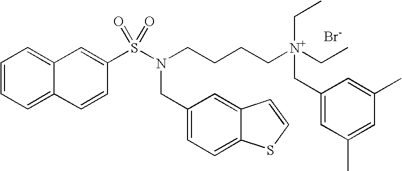 |
| 385 | 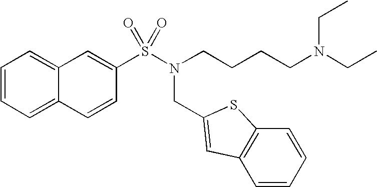 |
| 386 | 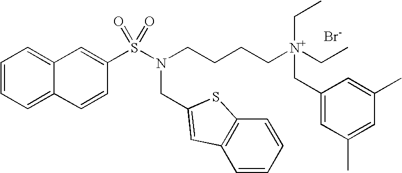 |
| 387 | 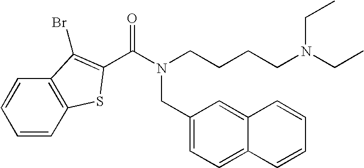 |
| 388 | 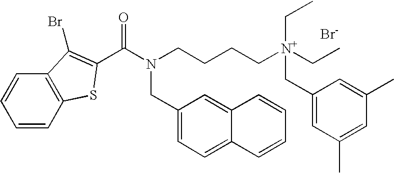 |
| 389 | 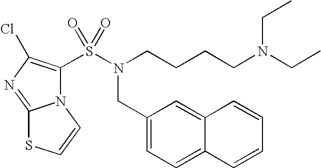 |
| 390 | 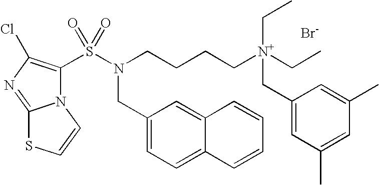 |
| 391 | 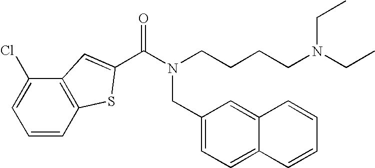 |
| 392 | 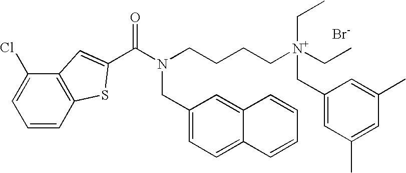 |
| 393 | 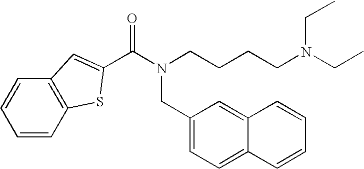 |
| 394 | 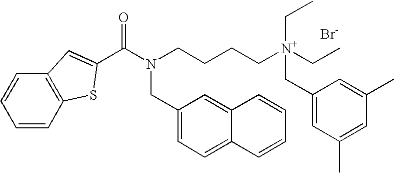 |
| 395 | 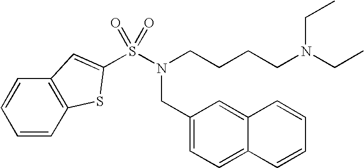 |
| 396 | 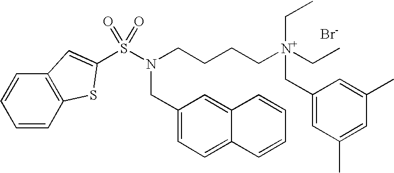 |
| 397 | 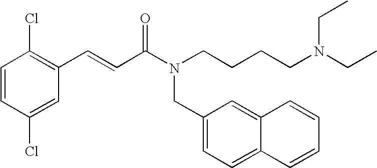 |
| 398 | 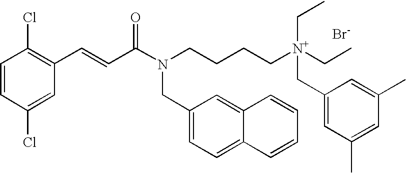 |
| 399 | 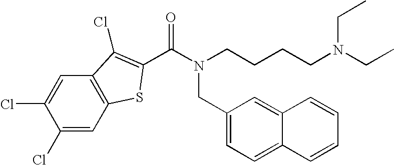 |
| 400 | 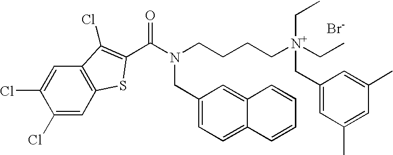 |
| 401 | 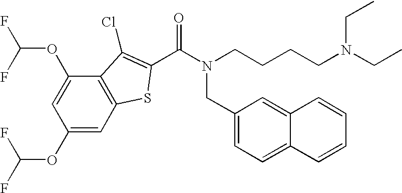 |
| 402 | 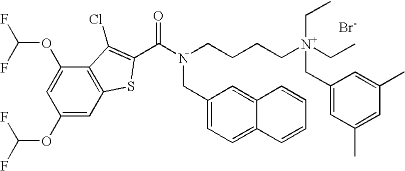 |
| 403 | 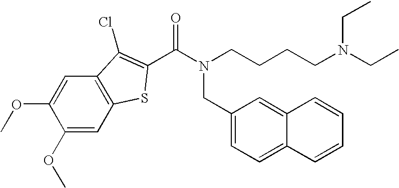 |
| 404 | 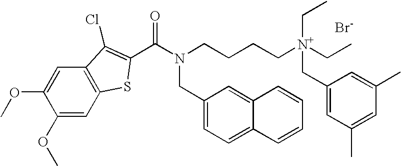 |
| 405 | 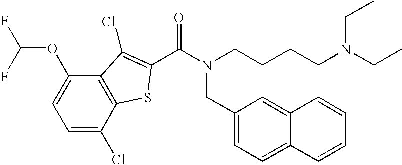 |
| 406 | 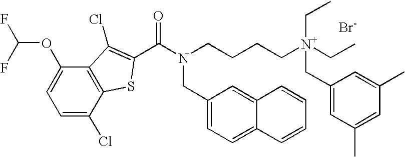 |
| 407 | 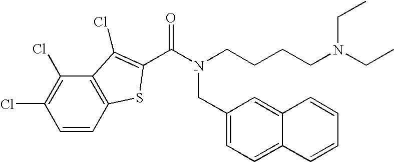 |
| 408 | 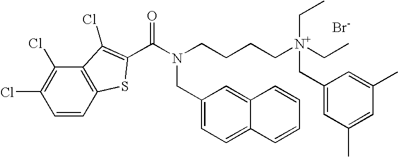 |
| 409 | 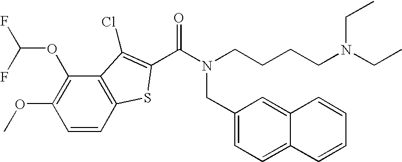 |
| 410 | 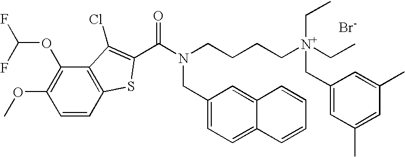 |
| 411 | 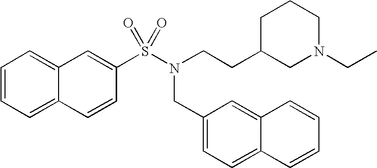 |
| 412 | 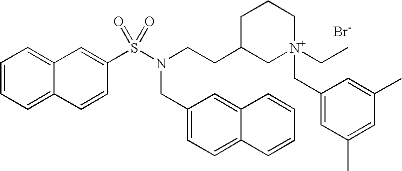 |
| 413 | 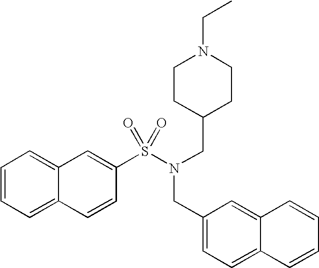 |
| 414 | 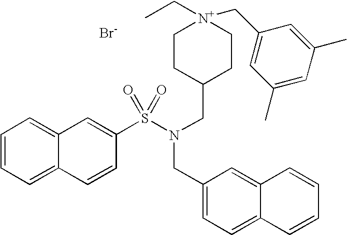 |

All quaternary ammonium cations in the preceding list are associated with a counteranion that is halide. However, the counteranion may be any other suitable anion, e.g., halogen, carboxylate or sulfonate.

3. Agrochemical compositions and use. Active compounds of the present invention can be used to prepare agrochemical compositions and used to control fungi in like manner as other antifungal compounds. See, e.g., U.S. Pat. No. 6,617,330; see also U.S. Pat. Nos. 6,616,952; 6,569,875; 6,541,500, and 6,506,794.

Active compounds described herein can be used for protecting plants against diseases that are caused by fungi. For the purposes herein, oomycetes shall be considered fungi. The active compounds can be used in the agricultural sector and related fields as active ingredients for controlling plant pests. The active compounds can be used to inhibit or destroy the pests that occur on plants or parts of plants (fruit, blossoms, leaves, stems, tubers, roots) of different crops of useful plants, optionally while at the same time protecting also those parts of the plants that grow later e.g. from phytopathogenic micro-organisms.

Active compounds may be used as dressing agents for the treatment of plant propagation material, in particular of seeds (fruit, tubers, grains) and plant cuttings (e.g. rice), for the protection against fungal infections as well as against phytopathogenic fungi occurring in the soil.

The active compounds may be used, for example, against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they may also be used against the Ascomycetes classes (e.g. Venturia and Erysiphe, Podosphaera, Monilinia, Uncinula) and of the Oomycetes classes (e.g. Phytophthora, Pythium, Plasmopara). Specific examples of fungi that may be treated include, but are not limited to, Septoria tritici, Stagonospora nodorum, Phytophthora infestans, Botrytis cinerea, Sclerotinia homoeocarpa and Puccinia recondita.

Target crops to be protected with active compounds and compositions of the invention typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fiber plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamon, camphor) or plants such as tobacco, nuts, coffee, eggplants, sugar cane, tea, pepper, vines including grape-bearing vines, hops, bananas, turf and natural rubber plants, as well as ornamentals (flowers, shrubs, broad-leafed trees and evergreens, such as conifers). This list does not represent any limitation.

The active compounds can be used in the form of compositions and can be applied to the crop area or plant to be treated, simultaneously or in succession with further compounds.

These further compounds can be e.g. fertilizers or micronutrient donors or other preparations which influence the growth of plants. They can also be selective herbicides as well as insecticides, fungicides, bactericides, nematicides, molluscicides, plant growth regulators, plant activators or mixtures of several of these preparations, if desired together with further carriers, surfactants or application promoting adjuvants customarily employed in the art of formulation.

The active compounds can be mixed with other fungicides, resulting in some cases in unexpected synergistic activities.

Mixing components which are particularly preferred are azoles such as azaconazole, bitertanol, propiconazole, difenoconazole, diniconazole, cyproconazole, epoxiconazole, fluquinconazole, flusilazole, flutriafol, hexaconazole, imazalil, imibenconazole, ipconazole, tebuconazole, tetraconazole, fenbuconazole, metconazole, myclobutanil, perfurazoate, penconazole, bromuconazole, pyrifenox, prochloraz, triadimefon, triadimenol, triflumizole or triticonazole; pyrimidinyl carbinoles such as ancymidol, fenarimol or nuarimol; 2-amino-pyrimidine such as bupirimate, dimethirimol or ethirimol; morpholines such as dodemorph, fenpropidin, fenpropimorph, spiroxamin or tridemorph; anilinopyrimidines such as cyprodinil, pyrimethanil or mepanipyrim; pyrroles such as fenpiclonil or fludioxonil; phenylamides such as benalaxyl, furalaxyl, metalaxyl, R-metalaxyl, ofurace or oxadixyl; benzimidazoles such as benomyl, carbendazim, debacarb, fuberidazole or thiabendazole; dicarboximides such as chlozolinate, dichlozoline, iprodine, myclozoline, procymidone or vinclozolin; carboxamides such as carboxin, fenfuram, flutolanil, mepronil, oxycarboxin or thifluzamide; guanidines such as guazatine, dodine or iminoctadine; strobilurines such as azoxystrobin, kresoxim-methyl, metominostrobin, pyraclostrobin, picoxystrobin, SSF-129, methyl 2[(2-trifluoromethyl)-pyrid-6-yloxymethyl]-3-methoxy-acrylate or 2-[{.alpha.[(.alpha.-methyl-3-trifluoromethyl-benzyl)imino]-oxy}-o-tolyl]-glyoxylic acid-methylester-O-methyloxime (trifloxystrobin); dithiocarbamates such as ferbam, mancozeb, maneb, metiram, propineb, thiram, zineb or ziram; N-halomethylthio-dicarboximides such as captafol, captan, dichlofluanid, fluoromide, folpet or tolyfluanid; copper compounds such as Bordeaux mixture, copper hydroxide, copper oxychloride, copper sulfate, cuprous oxide, mancopper or oxine-copper; nitrophenol derivatives such as dinocap or nitrothal-isopropyl; organo phosphorous derivatives such as edifenphos, iprobenphos, isoprothiolane, phosdiphen, pyrazophos or toclofos-methyl; and other compounds of diverse structures such as acibenzolar-S-methyl, harpin, anilazine, blasticidin-S, chinomethionat, chloroneb, chlorothalonil, cymoxanil, dichlone, diclomezine, dicloran, diethofencarb, dimethomorph, dithianon, etridiazole, famoxadone, fenamidone, fentin, ferimzone, fluazinam, flusulfamide, fenhexamid, fosetyl-aluminium, hymexazol, kasugamycin, methasulfocarb, pencycuron, phthalide, polyoxins, probenazole, propamocarb, pyroquilon, quinoxyfen, quintozene, sulfur, triazoxide, tricyclazole, triforine, validamycin, (S)-5-methyl-2-methylthio-5-phenyl-3-phenylamino-3,5-di-hydroimidazol-4-one (RPA 407213), 3,5-dichloro-N-(3-chloro-1-ethyl-1-methyl-2-oxopropyl)-4-methylbenzamide (RH-7281), N-allyl-4,5-dimethyl-2-trimethylsilylthiophene-3-carboxamide (MON 65500), 4-chloro-4-cyano-N,N-dimethyl-5-p-tolylimidazole-1-sulfon-amide (IKF-916), N-(1-cyano-1,2-dimethylpropyl)-2-(2,4-dichlorophenoxy)-propionamide (AC 382042) or iprovalicarb (SZX 722).

The active compounds can be mixed with one or more systemically acquired resistance inducer (“SAR” inducer), alone or in combination with a fungicide as above. SAR inducers are known and described in, for example, U.S. Pat. No. 6,919,298. In general, a SAR inducer is any compound which has the ability to turn on resistance in a plant to a disease-causing agent, including, but not limited to a virus, a bacterium, a fungus, or combinations of these agents. In addition, an SAR inducer may induce resistance to insect feeding in a plant, as defined by Enyedi et al. (1992; Cell 70: 879-886). Exemplary SAR inducers cover many structural families of compounds, but are united by their ability to induce a resistance to plant diseases and/or pest feeding. One class of SAR inducers is the salicylates. The commercial SAR inducers acibenzolar-s-methyl (available as Actigard® from Syngenta), harpin protein (available as Messenger™ from Eden Biosciences), yeast extract hydrolysate from Saccharomyces cerevisiae (available as Keyplex® 350-DP® from Morse Enterprises Limited, Inc. of Miami, Fla.), and Oryzemate are useful in the present invention. Elicitors, including the Goemar products are another class of SAR inducers that can also be used. In addition, ethylene, its biosynthetic precursors, or ethylene releasing compounds such as Ethrel are considered SAR inducers of utility in this context. See also U.S. Pat. No. 6,919,298.

Suitable carriers and adjuvants can be solid or liquid and are substances useful in formulation technology, e.g. natural or regenerated mineral substances, solvents, dispersants, wetting agents, tackifiers, thickeners, binders or fertilizers.

A preferred method of applying an active compound of the invention, or an agrochemical composition which contains at least one of said compounds, is foliar application. The frequency of application and the rate of application will depend on the risk of infestation by the corresponding pathogen. However, the active compounds can also penetrate the plant through the roots via the soil (systemic action) by drenching the locus of the plant with a liquid formulation, or by applying the compounds in solid form to the soil, e.g. in granular form (soil application). In crops of water such as rice, such granulates can be applied to the flooded rice field. The active compounds may also be applied to seeds (coating) by impregnating the seeds or tubers either with a liquid formulation of the fungicide or coating them with a solid formulation.

The term locus as used herein is intended to embrace the fields on which the treated crop plants are growing, or where the seeds of cultivated plants are sown, or the place where the seed will be placed into the soil. The term seed is intended to embrace plant propagating material such as cuttings, seedlings, seeds, and germinated or soaked seeds.

The active compounds are used in unmodified form or, preferably, together with the adjuvants conventionally employed in the art of formulation. To this end they are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomizing, dusting, scattering, coating or pouring, are chosen in accordance with the intended objectives and the prevailing circumstances.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The formulation, i.e. the compositions containing the compound of formula I and, if desired, a solid or liquid adjuvant, are prepared in known manner, typically by intimately mixing and/or grinding the compound with extenders, e.g. solvents, solid carriers and, optionally, surface active compounds (surfactants).

Suitable carriers and adjuvants may be solid or liquid and correspond to the substances ordinarily employed in formulation technology, such as, e.g. natural or regenerated mineral substances, solvents, dispersants, wetting agents, tackifiers, thickeners binding agents or fertilizers. Such carriers are for example described in WO 97/33890.

Further surfactants customarily employed in the art of formulation are known to the expert or can be found in the relevant literature.

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

The compositions may also contain further adjuvants such as stabilizers, antifoams, viscosity regulators, binders or tackifiers as well as fertilizers, micronutrient donors or other formulations for obtaining special effects.

4. Technical materials. The compounds and combinations of the present invention may also be used in the area of controlling fungal infection (particularly by mold and mildew) of technical materials, including protecting technical material against attack of fungi and reducing or eradicating fungal infection of technical materials after such infection has occurred. Technical materials include but are not limited to organic and inorganic materials wood, paper, leather, natural and synthetic fibers, composites thereof such as particle board, plywood, wall-board and the like, woven and non-woven fabrics, construction surfaces and materials, cooling and heating system surfaces and materials, ventilation and air conditioning system surfaces and materials, and the like. The compounds and combinations according to the present invention can be applied to such materials or surfaces in an amount effective to inhibit or prevent disadvantageous effects such as decay, discoloration or mold in like manner as described above. Structures and dwellings constructed using or incorporating technical materials in which such compounds or combinations have been applied are likewise protected against attack by fungi.

5. Pharmaceutical uses. In addition to the foregoing, active compounds of the present invention can be used in the treatment of fungal infections of human and animal subjects (including but not limited to horses, cattle, sheep, dogs, cats, etc.) for medical and veterinary purposes. Examples of such infections include but are not limited to ailments such as Onychomycosis, sporotichosis, hoof rot, jungle rot, Pseudallescheria boydii, scopulariopsis or athletes foot, sometimes generally referred to as “white-line” disease, as well as fungal infections in immunocomprised patients such as AIDS patients and transplant patients. Thus, fungal infections may be of skin or of keratinaceous material such as hair, hooves, or nails, as well as systemic infections such as those caused by Candida spp., Cryptococcus neoformans, and Aspergillus spp., such as in pulmonary aspergillosis and Pneumocystis carinii pneumonia. Active compounds as described herein may be combined with a pharmaceutically acceptable carrier and administered or applied to such subjects or infections (e.g., topically, parenterally) in an amount effective to treat the infection in accordance with known techniques, as (for example) described in U.S. Pat. Nos. 6,680,073; 6,673,842; 6,664,292; 6,613,738; 6,423,519; 6,413,444; 6,403,063; and 6,042,845; the disclosures of which applicants specifically intend be incorporated by reference herein in their entirety.

In addition to the foregoing, the compounds may be used for the treatment of obesity, metabolic syndrome, atherosclerosis, cardiovascular disease and insulin resistance, e.g., type II or adult-onset diabetes, in human or animal subjects. The compounds of this invention may also be used in conjunction with other pharmaceutical agents for the treatment of the diseases/conditions described herein, as described hereinabove and below. In combination therapy treatment, both the compounds of this invention and the other drug therapies are administered to mammals (e.g., humans, male or female) by conventional methods. Representative examples of second compounds in the combination aspect of this invention included, but are not limited to, any antiatherosclerosis agent, cholesterol absorption inhibitor, HMG-CoA reductase inhibitor, MTP/Apo B secretion (microsomal triglyceride transfer protein and/or apolipoprotein B secretion) inhibitor, or HMG-CoA synthase inhibitor. Examples of other second compounds are described in US 2003/0187254A1.

“Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically-acceptable carrier” as used herein means a pharmaceutically-acceptable material, composition or vehicle, such as a liquid or solid filler, diluent, excipient, solvent or encapsulating material, involved in carrying or transporting the subject peptidomimetic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the active ingredient which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a peptide or peptidomimetic of the present invention with liquid carriers, or finely divided solid carriers, or both, and then, if necessary, shaping the product.

The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Pharmaceutical compositions of this invention suitable for parenteral administration comprise one or more active compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given by any suitable means of administration including orally, parenterally, topically, transdermally, rectally, etc. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Topical or parenteral administration is preferred.

“Parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

Actual dosage levels of the active ingredients in the pharmaceutical compositions of this invention may be varied so as to obtain an amount of the active ingredient which is effective to achieve the desired therapeutic response, e.g., antimycotic activity, for a particular patient, composition, and mode of administration, without being toxic to the patient. The selected dosage level will depend upon a variety of factors including the activity of the particular active compound employed, the route of administration, the time of administration, the rate of excretion of the particular active compound being employed, the duration of the treatment, other drugs, compounds and/or materials used in combination with the particular inhibitor employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. As a general proposition, a dosage from about 0.01 or 0.1 to about 50, 100 or 200 mg/kg will have therapeutic efficacy, with all weights being calculated based upon the weight of the active compound, including the cases where a salt is employed.

The present invention is explained in greater detail in the following non-limiting Examples.

### EXAMPLE 1

### Diethyl(3,5-dimethylbenzyl)[bis-(3,5-dimethylbenzyl)aminobutyl]ammonium formate (Compound 28)

A mixture of 29 mg (0.2 mmol) of diethylaminobutylamine, 119 mg (0.6 mmol) of 3,5-dimethylbenzyl bromide, and 0.6 mL (0.6 mmol) of 1M NaHCO₃, and 2 mL of THF in an 8 mL sealed vial was shaken at room temperature for 22 hr. The reaction product mixture was syringe filtered, and then purified by preparative HPLC using acetonitrile/0.05% aqueous formic acid. Lyophilization of the HPLC fractions gave 48 mg (0.09 mmol) of diethyl(3,5-dimethylbenzyl)[bis-(3,5-dimethylbenzyl)-aminobutyl]-ammonium formate as a gum. ¹H NMR (CDCl₃): δ1.39 (t, 6H), 1.55 (br m, 2), 1.75 (br m, 2), 2.29 (s, 12), 2.31 (s, 6), 3.45 (s, 4), 4.50 (s, 2), 6.88-6.95, (m, 9), and 8.68 ppm (s, 1). MS m/z: 499.4 (M⁺).

### EXAMPLE 2

### N-4-Bromobenzyl-N-[bis-(4-bromobenzyl)aminopropyl]morpholinium formate (Compound 29)

A mixture of 14 mg (0.1 mmol) of N-3-aminopropylmorpholine, 75 mg (0.3 mmol) of 4-bromobenzyl bromide, and 0.3 mL (0.3 mmol) of 1M NaHCO₃, and 1 mL of THF in an 8 mL sealed vial was shaken at room temperature for 22 hr. The reaction product mixture was syringe filtered, and then purified by preparative HPLC using acetonitrile/0.05% aqueous formic acid. Lyophilization of the HPLC fractions gave 11.5 mg (0.016 mmol) of N-4-bromobenzyl-N-[bis-(4-bromobenzyl)-aminopropyl]-morpholinium formate as a gum. ¹H NMR (CDCl₃): δ1.96 (br m, 2), 2.55 (br t, 2), 3.35 (br m, 4), 3.51 (s, 4), 3.70 (br t, 4), 3.98 (br t, 2), 5.04 (br s, 2), 7.14 (d, 4), 7.32 (d, 2), 7.42 (d, 4), 7.50 (d, 2), and 8.80 ppm (s, 1). MS m/z: 653.0 (M⁺).

### EXAMPLE 3

### N-[6-Bromo-2-(3,5-dimethylbenzylthio)quinazoin-4-on-3-propyl]-N-3,5-dimethylbenzylmorpholinium formate (Compound 18)

A mixture of 28 mg (0.1 mmol) of 4-bromo-2-methoxycarbonylphenyl isothiocyanate and 16 mg (0.11 mmol) of N-3-aminopropylmorpholine in 1 mL of THF was heated in an 8 mL sealed vial at 60° C. for 3 hrs. The reaction was cooled and then 30 mg (0.15 mmol) of 3,5-dimethylbenzyl bromide and 0.12 mL (0.12 mmol) of 1M NaHCO₃ was added. The reaction mixture was heated at 60° C. for another 3 hrs. The reaction product mixture was syringe filtered, and then purified by preparative HPLC using acetonitrile/0.05% aqueous formic acid. Lyophilization of the HPLC fractions gave 4 mg (0.006 mmol) of N-[6-bromo-2-(3,5-dimethylbenzylthio)quinazolin-4-on-3-propyl]-N-3,5-dimethylbenzylmorpholinium formate as a gum. ¹H NMR (CDCl₃): δ2.26 (s, 6), 2.29 (s, 6), 2.39 (br m, 2), 3.60 (br m, 2), 3.79 (br m, 4), 4.04 (br m, 4), 4.24 (br m,2), 4.47 (s, 2), 4.83 (br m, 2), 6.90 (d, 1), 7.04 (d, 2), 7.47 (d, 1) 7.79 (d of d, 1), 8.24 (d, 1) and 8.74 ppm (s, 1). MS m/z: 622.1 (M⁺). 6-Bromo-2-(3,5-dimethylbenzylthio)-3-(N-morpholinopropyl)-quinazolin-4-one was also isolated as a minor product.

### EXAMPLE 4

### 2-(3,5-Dimethylbenzylthio)-3-(3-methylsulfonyloxypropyl)-6,7,8-trimethoxyquinazolin-4-one

A mixture of 113 mg (0.4 mmol) of 2-methoxycarbonyl-4,5,6-trimethoxyphenyl isothiocyanate and 61 μL (60 mg, 0.8 mmol) of 3-aminopropanol in 4 mL of THF was heated in an 8 mL sealed vial at 60° C. for 4 hrs. The reaction mixture was cooled and 80 mg (0.4 mmol) of 3,5-dimethylbenzyl bromide and 0.4 mL of 1M NaCO₃ was added. The reaction mixture was stirred overnight at room temperature, and then poured into toluene and water. The toluene layer was separated, washed with water, and dried by filtration through phase separating paper. The residue after solvent removal was digested with toluene, concentrated, and the solid was separated to give 0.18 gm (0.4 mmol) of 2-(3,5-dimethylbenzylthio)-3-(3-hydroxypropyl)-6,7,8-trimethoxyquinazolin-4-one.

To 179 mg (0.4 mmol) of 2-(3,5-dimethylbenzylthio)-3-(3-hydroxypropyl)-6,7,8-trimethoxyquinazolin-4-one in 15 mL of dichloromethane at −20° C. was added 167 μL (121 mg, 1.2 mmol) of triethylamine followed by 77 μL (114 mg, 1.0 mmol) of methanesulfonyl chloride. After 0.5 hr, the reaction mixture was warmed to room temperature, and then was poured into dichloromethane and water. The dichloromethane layer was separated, washed several times with water, and then passed through phase separating paper. The solvent was removed in vacuo, and the residue was digested with hexane. The white solid product was collected by filtration to yield 159 mg (0.30 mmol) of 2-(3,5-dimethylbenzylthio)-3-(3-methylsulfonyloxypropyl)-6,7,8-trimethoxyquinazolin-4-one. ¹H NMR (CDCl₃): δ2.24 (m, 2), 2.30 (s, 6), 3.01 (s, 3), 4.25 (t, 2), 4.32 (t, 2), 4.49 (s, 2), 6.91 (d, 1), 7.08 (d, 2), and 7.36 ppm (s, 1). MS m/z: 523.1 (M+H).

### EXAMPLE 5

### 2-(3,5-Dimethylbenzylthio)-3-[3-(4-methylpiperazinyl)propyl]-6,7,8-trimethoxyquinazolin-4-one (Compound 9)

A mixture of 54 mg (0.1 mmol) of 2-(3,5-dimethylbenzylthio)-3-(3-methylsulfonyloxypropyl)-6,7,8-trimethoxyquinazolin-4-one and 30 mg (0.3 mmol) of N-methylpiperazine in 1.2 mL of dioxane was heated in an 8 mL sealed vial for 18 hrs. The mixture was cooled and poured in to toluene and water. The toluene layer was separated, washed with water, and dried by passing through phase separating paper. Purification by preparative HPLC using acetonitrile/0.05% aqueous formic acid gave 21 mg (0.04 mmol) of 2-(3,5-dimethylbenzylthio)-3-[3-(4-methylpiperazinyl)propyl]-6,7,8-trimethoxyquinazolin-4-one. ¹H NMR (CDCl₃): δ1.95 (br m, 2), 2.29 (s, 3), 2.31 (s, 6), 2.47 (br m, 4), 3.94 (s, 3), 4.02 (s, 3), 4.06 (s, 3), 4.17 (br m, 2), 4.47 (s, 2), 6.92 (d, 1), 7.08 (d, 2), and 7.38 ppm (s, 1). MS m/z: 527.2 (M⁺).

### EXAMPLE 6

### 4-[2-(3,5-Dimethylbenzylthio)-6,7,8-trimethoxyquinazolin-4-onyl-3-propyl]-1-methyl-1-(3,5-dimethylbenzyl)piperazinylium formate (Compound 10)

To 11 mg (0.02 mmol) of 2-(3,5-dimethylbenzylthio)-3-[3-(4-methyl-piperazinyl)propyl]-6,7,8-trimethoxyquinazolin-4-one in 1 mL of dioxane was added 4 mg (0.02 mmol) of 3,5-dimethylbenzyl bromide and 4.2 μL (3 mg, 0.03 mmol) of triethylamine. The mixture was heated to 80° C. for 20 hrs. The alkylation product was purified by preparative HPLC using acetonitrile/0.05% aqueous formic acid to give 5 mg of the quaternary ammonium salt, 4-[2-(3,5-dimethylbenzylthio)-6,7,8-trimethoxyquinazolin-4-onyl-3-propyl]-1-methyl-1-(3,5-dimethylbenzyl)piperazinylium formate. ¹H NMR (CDCl3): δ1.95 (br m, 2), 2.29 (s, 6), 2.34 (s, 6), 3.96 (s, 3), 4.02 (s, 3), 4.09 (s, 3), 4.19 (br t, 2), 4.48 (s, 2), 4.64 (br m, 2), 6.91 (d, 1), 7.05 (d, 2), 7.09 (d, 3), 7.37 (s, 1), and 8.74 ppm (s, 1). MS m/z: 645.3 (M⁺).

### EXAMPLE 7

### Diethyl(3,5-dimethylbenzyl)[N-(3,5-dimethylbenzyl)-2-naphthalenecarboxamidobutyl]ammonium bromide (Compound 133)

A solution of 209 mg (1.56 mmol) of 3,5-dimethylbenzaldehyde and 204 mg (1.42 mmol) of 4-(diethylamino)butylamine in 25 mL of methanol was stirred at room temperature overnight. To this solution was added 94 mg (2.49 mmol) of sodium borohydride. After 1 hr of stirring, the mixture was poured into water and acidified with 5% aqueous hydrochloric acid. The aqueous solution was extracted with diethyl ether three times, and the ether extracts were discarded. The aqueous fraction was basified with saturated sodium bicarbonate, and then extracted thoroughly with diethyl ether to give 185 mg (0.71 mmol) of N-[4-(diethylamino)butyl]-N-(3,5-dimethylbenzyl)amine.

To a mixture of 60 mg (0.23 mmol) of N-[4-(diethylamino)butyl]-N-(3,5-dimethylbenzyl)amine, 45 mg (0.25 mmol) of 2-naphthoic acid, and 48 μL (0.27 mmol) of diisopropylethylamine in 1 mL of anhydrous DMF at room temperature was added 96 mg (0.25 mmol) of HATU [O-(7-azabenzotriazol-1-yl)-N,N,N′,N′-tetramethyluronium hexafluorophosphate]. After the mixture was stirred for 5 hr, it was diluted with ethyl acetate. The solution was washed with water four times, then with saturated sodium chloride, and finally was dried with sodium sulfate. The solvent was removed by rotoevaporation, and the crude product was purified by preparative TLC to give 92 mg (0.22 mmol) of N-(4-diethylaminobutyl)-N-(3,5dimethylbenzyl)-2-naphthoic acid amide as a mixture of rotamers. ¹H NMR (CDCl3): δ1.31 (t, 6), 2.29 (s, 6), 3.18 (m, 6), 3.49 (m, 2), 4.50 (s, 2), 6.74 (s, 2), and 6.91 ppm (s, 1). MS m/z: 417.3 (M⁺).

The 47 mg (0.11 mmol) of mixed rotamers of N-(4-diethylaminobutyl)-N-(3,5dimethylbenzyl)-2-naphthoic acid amide and 33 mg (0.17 mmol) of 3,5-dimethylbenzyl bromide in 1.3 mL 0f anhydrous acetonitrile was added 35 mg (0.42 mmol) of sodium bicarbonate. The mixture was stirred at 65° C. for 3 days, and the product was purified by preparative TLC to give 18 mg (0.03 mmol) of diethyl(3,5-dimethylbenzyl)-[N-(3,5-dimethylbenzyl)-2-naphthalenecarboxamidobutyl]ammonium bromide. ¹H NMR (CDCl3): δ2.26 (s, 6), 2.30 (s, 6), 3.21 (m, 6), 3.49 (m, 2), 4.24 (s, 2), 4.54 (s, 2), 6.74 (s, 2), 6.88 (s, 1), 6.93 (s, 2), and 7.08 ppm (s, 1). MS m/z: 535.3 (M⁺-Br)).

### EXAMPLE 8

### N-(4-Diethylaminobutyl)-N-(3,5-dimethylbenzyl)-2-naphthalenesulfonamide (Compound 105)

To a mixture of 65 mg (0.25 mmol) of N-[4-(diethylamino)butyl]-N-(3,5-dimethylbenzyl)amine (see Example 7) and 129 μL (0.74 mmol) of diisopropylethylamine in 5 mL of anhydrous dichloromethane at room temperature was added 56 mg (0.25 mmol) of 2-naphthalenesulfonyl chloride. After the mixture was stirred for 3 hr, it was diluted with ethyl acetate. The solution was washed with water several times, then with saturated sodium chloride, and finally was dried with sodium sulfate. The solvent was removed by rotoevaporation, and the crude product was purified by preparative TLC to give 70 mg (0.15 mmol) of N-(4-diethylaminobutyl)-N-(3,5dimethylbenzyl)-2-naphthalenesulfonamide. ¹H NMR (CDCl3): δ1.13 (t, 6), 2.20 (s, 6), 3.20 (t,2), 4.29 (s, 2), 6.79 (s, 2), and 6.86 ppm (s, 1). MS m/z: 453.2 (M⁺).

### EXAMPLE 9

### Diethyl(3,5-dimethylbenzyl)[N-(3,5-dimethylbenzyl)-2-naphthalenesulfonamidobutyl]ammonium bromide (Compound 85)

A mixture of 37 mg (0.082 mmol) of N-(4-diethylaminobutyl)-N-(3,5dimethylbenzyl)-2-naphthalenesulfonamide (Compound 85), 25 mg (0.12 mmol) of 3,5-dimethylbenzyl bromide, and 20 mg (0.24 mmol) of sodium bicarbonate in 0.8 mL of anhydrous acetonitrile was stirred at 65° C. for 48 hr. The solution was concentrated to dryness, and the crude product was triturated with 30% ether/hexane to remove excess bromide and give 10 mg (0.015 mmol) of diethyl(3,5-dimethylbenzyl)-[N-(3,5-dimethylbenzyl)-2-naphthalenesulfonamidobutyl]ammonium bromide. ¹H NMR (CDCl3): δ1.43 (t, 6), 2.16 (s, 6), 2.34 (s, 6), 4.25 (s, 2), 4.48 (s, 2), 6.80 (s, 2), 6.85 (s, 1), 7.01 (s, 2), and 7.10 ppm (s, 1). MS m/z: 571.3 (M⁺-Br).

### EXAMPLE 10

### N-(4-Diethylaminobutyl)-N-(2-naphthylmethyl)-2-naphthalenesulfonamide (Compound 199)

A solution of 370 mg (2.37 mmol) of 2-naphthaldehyde and 326 mg (2.26 mmol) of 4-(diethylamino)butylamine in 5 mL of methanol was stirred at room temperature overnight. The solution was cooled to 0° C., and 233 mg (6.16 mmol) of sodium borohydride was added. After 1 hr of stirring, aqueous sodium hydroxide (1 mL) was added to the mixture. The product was extracted with diethyl ether several times, and the combined ether extracts were dried with sodium sulfate. The ether was removed by rotoevaporation to give 545 mg (1.92 mmol) of N-[4-(diethylamino)butyl]-N-(2-naphthylmethyl)amine.

To a mixture of 50 mg (0.175 mmol) of N-[4-(diethylamino)butyl]-N-(2-naphthylmethyl)amine and 91 μL (0.18 mmol) of diisopropylethylamine in 1 mL of anhydrous dichloromethane at room temperature was added 41 mg (0.18 mmol) of 2-naphthalenesulfonyl chloride. After the mixture was stirred overnight, it was diluted with ethyl acetate. The solution was washed with water several times, then with saturated sodium chloride, and finally was dried with sodium sulfate. The solvent was removed by rotoevaporation, and the crude product was purified by preparative TLC to give 54 mg (0.11 mmol) of N-(4-diethylaminobutyl)-N-(2-naphthylmethyl)-2-naphthalenesulfon-amide. MS m/z: 475.2 (M⁺).

### EXAMPLE 11

### Diethyl(3,5-dimethylbenzyl)[N-(2-naphthylmethyl)-2-naphthalenesulfonamidobutyl]ammonium bromide (Compound 200)

A mixture of 35 mg (0.074 mmol) of N-(4-diethylaminobutyl)-N-(2-naphthylmethyl)-2-naphthalenesulfonamide, 26 mg (0.13 mmol) of 3,5-dimethylbenzyl bromide, and 90 mg (1.07 mmol) of sodium bicarbonate in 1.0 mL of anhydrous acetonitrile was stirred at 65° C. for 48 hr. The solution was concentrated to dryness, and dichloromethane was added to the crude product. The mixture was filtered, and the mother liquor was concentrated to dryness. The residue was triturated with ether to remove excess bromide and give 14 mg (0.021 mmol) of diethyl(3,5-dimethylbenzyl)[N-(2-naphthylmethyl)-2-naphthalene-sulfonamidobutyl]ammonium bromide. ¹H NMR (CDCl3): δ1.21 (m, 6, 2.31 (s, 6), 4.21 (s, 2), 4.48 (s, 2), 6.90 (s, 2), 7.08, and 8.45 ppm (s, 1). MS m/z: 593.5 (M⁺-Br).

### EXAMPLE 12

### In Vivo and In Vitro Assays

Fungicidal activity for the compounds described in this invention was determined using a microtiter plate format. In primary screening, test compounds in 1 μL of dimethylsulfoxide (DMSO) are delivered to individual wells of a 96-well microtiter plate. Then 100 μL of minimal media consisting of 1.5% agar is delivered to each well and allowed to cool. Finally, inoculation is carried out by the addition of 10 μL of an aqueous suspension of fungal spores to the surface of the solid agar. The plates are covered and incubated in a controlled environment at 20° C. Fungicidal activity is determined by visual inspection and photometric analysis of fungal growth after 3-5 days, depending on the pathogen. Commercial standards (azoxystrobin, benomyl, captan, chlorothalonil, famoxadone, flusilazole, and propiconazole) are included in all assays. Test pathogens include Septoria tritici, Stagonospora nodorum, Phytophthora infestans, and Botrytis cinerea. Dose response data for compounds found to be fungicidal in primary screening are obtained by screening 3-fold serial dilutions of the test compound. Fungicidal activity, noted as IC50 values in μM concentration, for certain of the compounds covered in this invention is included in the following Table 1. In vitro IC50 values of Ustilago maydis ACC were also determined by standard procedures. For example, Ustilago maydis ACC was cloned, expressed, and purified as described (Weatherly et al., (2004) Biochemical J. 380:105-110). A K53R site-directed mutation, which confers resistance to soraphen (Yang et al., (2004) Molecular Cell 16:881-891) was introduced into the coding sequence using a Quick-Change Site-Directed Mutagenesis Kit (Stratagene). ACC activity was measured by following the incorporation of [¹⁴C]-bicarbonate into malonyl-CoA essentially as described (Weatherly et al., (2004) Biochemical J. 380:105-110). Briefly, the reaction mixture (100 μL) contained 50 mM Hepes (pH8), 2.5 mM MgCl₂, 1 mM ATP, 0.5 mM DTT, 10 mM NaHCO₃, 0.95 mM NaH¹⁴CO₃ (48.7 mCi/mmol), 1% (v/v) DMSO (with or without inhibitor), and 0.33 mM acetyl-CoA. Reactions were initiated by the addition of 1.2 μg ACC. After 10 minutes at 30° C., the reaction was stopped by the addition of 20 μL concentrated HCl. The samples were evaporated by heating at 60° C., and resuspended in 250 μL H₂O. For quantification of incorporated label, the samples were counted after addition of 3 mL scintillation fluid. For inhibitor characterization, data were fit to dose-response curves and IC₅₀ values are reported (i.e. the concentration of inhibitor that gives 50% inhibition). Data from some representative compounds are shown in Tables I, II, and III. Of particular note, the K53R mutation that confers resistance to soraphen inhibition also confers resistance to inhibition by Compounds 28, 184, and 322 (Table III). This demonstrates that these compounds act at a site that overlaps with the soraphen binding pocket.

TABLE I
In vivo and in vitro inhibitory activity of representative compounds

| | | | | | In vitro |
|---|---|---|---|---|---|
| | In vivo IC50 (μM) | | | | IC50 (μM) |
| | B. | | | | U. maydis |
| Cmpd | cinerea | P. infestens | S. nodorum | S. tritici | ACC activity |
| 22 | 6.7 | 35 | 4.6 | 4.4 | 12 |
| 15 | 2 | 18 | 8 | 4 | 16 |
| 7 | 4.3 | 200 | 8 | 4.8 | 17 |
| 28 | 1.8 | 21.5 | 7.2 | 11 | 17 |
| 19 | 7 | 25 | 64 | 22 | 21 |
| 26 | 1 | 23.5 | 5 | 6 | 39 |
| 25 | 1.4 | 18.2 | 5.8 | 10 | 42 |

To test the effect of compounds against mammalian ACC both human and rat enzyme preparations were employed. ACC was purified from rat liver according to published protocols (Rubink and Winder, J Appl Physiol 98, 1221-1227 (2005); Trumble et al., Eur J Biochem 231, 192-198 (1995)). Rat liver contains both ACC1 and ACC2 in a ˜85:15 ratio, respectively (Harwood et al., J Biol Chem 278, 37099-37111 (2003)). ACC activity in the rat liver preparation was measured by following incorporation of [¹⁴C]bicarbonate into acetyl-CoA (Thumpy and Wakil, J Biol Chem 260, 6318-6323 (1985). This is essentially the same assay used for fungal ACC with the primary differences being that it is carried out at 37° C. at pH 7.5, and citrate is included since it is required for activation of mammalian ACC. Soraphen inhibited rat liver ACC activity with an IC50 of 14 nM similar to the 17 nM IC50 determined for inhibition of fungal ACC (Table II). Note that this assay is performed under tight binding conditions with a nominal enzyme concentration of 25 nM. Therefore, this data is in good agreement with the FP saturation binding assay where we determined that rat liver ACC binds soraphen with a K_{d} of 3.2 nM. Table II also shows comparative inhibition data for several representative compounds against both fungal and rat liver ACC. These results demonstrate that compounds in this claim inhibit mammalian ACC in addition to fungal ACC, and that specificity can be achieved at this site.

For human ACC preparations we cloned full-length cDNAs for both the human isoforms (hACC1 and hACC2) and expressed each as a recombinant protein using a baculovirus system. Double stranded cDNA from human fetal liver (Quick-Clone™ cDNA, cat. #7171-1) was purchased from BD/Clontech (Mt. View, Calif.). PCR primers were designed for full-length hACC1 and hACC2 using recent GenBank submissions AY237919 and AJ575592, respectively. The Advantage® GC 2 PCR kit (BD/Clontech) was used for amplification. The PCR products (˜7 Kb) were TOPO-cloned into the pCR4Blunt vector (Invitrogen, Carlsbad, Calif.), and 6 clones of each gene were isolated. All clones had high error rates and the ACC2 clones showed evidence of alternately spliced mRNAs whose physiological significance is unknown. Full length versions of the two cDNAs corresponding to their respective Genbank sequences were assembled in pBluescript® SK+ (Stratagene, La Jolla, Calif.) by combining error-free fragments from the PCR clones. The resulting hACC1 clone, designated pCS35, has six silent nucleotide differences compared to Genbank accession AY237919. The final hACC2 clone, designated pCS36, has ten silent nucleotide differences compared to Genbank accession AJ575592. We initially attempted to express these cDNAs in E. coli by cloning both as NdeI/NotI fragments into the T7 expression vector pET30a (Novagen, Madison, Wis.) to produce C-terminal His-tagged fusion proteins. When E. coli expression failed, we designed primers to PCR amplify the cDNAs along with the in-frame 3′-his tag sequences from the pET vector and TOPO-cloned the resulting products into the Gateway® entry vector pENTRID-TOPO®. The ACC2 primers were designed to delete amino acids 1-148 and add Met-Gly in front of Lys149 in the final product. The resulting entry clones were transferred to BaculoDirect™ linear DNA (Invitrogen corp.) using the Gateway® LR recombination reaction. The recombinant baculovirus DNA was transfected into insect cells and viral amplification was performed according to the manufacturers protocols. For protein expression sf9 cells were infected with a P3 viral stock in the presence of 50 uM biotin. The cells were harvested after 48-72 hr, lysed by sonication, and the resulting extract was clarified by centrifugation. ACC in the crude extract was concentrated by ammonium sulfate precipitation (40% w/v) and purified by Ni²⁺-NTA chromatography (Novagen) followed by anion exchange chromatography on a UNO-Q column (BioRad). hACC1 and hACC2 activity was measured as described for rat liver ACC.

TABLE II
Inhibition of fungal and mammalian ACC by representative compounds

| | Enzyme Activity IC₅₀ (μM) | | | |
|---|---|---|---|---|
| | | | Human | Human |
| Cmpd | U. maydis ACC¹ | Rat liver ACC² | ACC1 | ACC2 |
| soraphen | 0.017 | 0.014 | 0.011 | 0.0027 |
| Cmpd 61 | 9 | ND³ | | |
| Cmpd 74 | 9 | ND | | |
| Cmpd 76 | 30 | 147 | | |
| Cmpd 81 | 13 | 49 | | |
| Cmpd 85 | 15 | 43 | | |
| Cmpd 133 | 3 | 45 | | |
| Cmpd 200 | 0.56 | 11 | 7.6 | 22 |
| Cmpd 237 | 1.4 | 17 | 12 | 50 |
| Cmpd 260 | 1.4 | | 2.8 | 11 |
| Cmpd 322 | 0.04 | | 0.25 | 1.0 |
| Cmpd 326 | 0.18 | | 0.15 | 1.5 |

¹enzyme concentration 50 nM
²enzyme concentration 25 nM
³ND = none detected

### Tryptophan Fluorescence Binding Assays

The soraphen binding pocket contains a tryptophan residue. Thus, tryptophan fluorescence may be used to directly monitor binding of a ligand to this site. For example, soraphen binding to recombinant yeast BC domains can be detected by an increase in tryptophan fluorescence (Yang et al. (2004) Molecular Cell 16:881-891). We conducted a similar assay in a 96-well format. Briefly, 200 nM protein in 100 mM Tris pH 8 containing 100 mM NaCl and 0.01% Tween 20 (v/v) was incubated in the presence of various amounts of test compounds dissolved in DMSO (1% v/v final concentration). Tryptophan fluorescence at 330 nM was measured in a Pherastar plate reader (BMG Labtech) using 280 nm excitation. Data were fit to dose-response curves and EC50 values are reported (i.e. the concentration of compound which gives a 50% effect on tryptophan fluorescence). Note that for conditions where the EC50 is significantly greater than 200 nM it is an estimate of the binding K_{d}. An example of this assay performed with soraphen is shown in Table III. Because soraphen binds to the yeast BC domain with a 1 nM K_{d}, this assay was performed under stoichiometric binding conditions and an EC50 of 100 nM was expected consistent with the experimental results. Additionally as expected, soraphen had no effect on the tryptophan fluorescence of a yeast BC domain containing a K73R mutation since this mutation confers resistance to soraphen binding. An example of this assay performed using Compound 28 is also shown in Table III. In this case, tryptophan fluorescence was quenched in the presence of this compound consistent with positive charge of Compound 28. The observed EC50 for tryptophan fluorescence quenching is in good agreement with the IC50 measured for ACC activity inhibition as expected. Finally, this compound had no effect on the tryptophan fluorescence of a yeast BC domain containing a K73R mutation consistent with the enzyme activity data, and providing independent conformation that this compound binds to a site that overlaps with the soraphen binding site.

TABLE III
Compound 28 inhibits ACC through interaction at the
soraphen binding site

| | | | Tryptophan fluorescence | |
|---|---|---|---|---|
| | Enzyme activity¹ IC50 (μM) | | binding assay² EC50 (μM) | |
| | U. maydis | U. maydis | Yeast | Yeast |
| cmpd | wt ACC | K53R ACC³ | wt BC | K73R BC³ |
| soraphen | 0.017 | ND³ | 0.09 | ND |
| Cmpd 28 | 11 | 52 | 11 | ND |

¹enzyme concentration = 50 nM
²protein concentration = 200 nM
³U. maydis K53 is equivalent to yeast K73
⁴ND = none detected

That which is claimed is:

1. A compound of formula I: 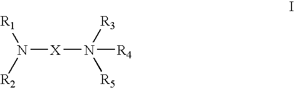
wherein
R₁ and R₂ are taken independently selected from the group consisting of H, alkyl, alkenyl, alkynyl, haloalkyl, haloalkenyl, haloalkynyl, COR₆, COOR₆, CONR₇R₈, SO₂NR₇R₈, SO₂R₉, alkoxyalkyl, alkylthioalkyl, aryl, arylalkyl or aryloxyalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, and NO₂,
or R₁ and R₂ may together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring;
R₃ and R₄ are each independently selected from the group consisting of alkyl, alkenyl, alkynyl, haloalkyl, haloalkenyl, haloalkynyl, CH₂COR₆, CH₂COOR₆, CH₂CONR₇R₈, O, aryl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, and NO₂,
or R₃ and R₄ may together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring;
R₅ can be a non-bonded pair of electrons or is selected from the group consisting of alkyl, alkenyl, alkynyl, haloalkyl, haloalkenyl, haloalkynyl; arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, and NO₂;
when R₅ is alkyl, alkenyl, alkynyl, haloalkyl, haloalkenyl, haloalkynyl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂, the N-atom to which R₃, R₄, and R₅ are attached will carry a positive charge and be associated with anion Y⁻, where Y may be halogen, carboxylate, sulfonate or any other suitable counterion;
R₆, R₇, R₈ and R₉ are each independently selected from the group consisting of H, alkyl, haloalkyl, alkoxyalkyl, haloalkoxyalkyl, alkylthioalkyl, haloalkylthioalkyl, aryl or arylalkyl optionally substituted with halogen, alkyl, haloalkyl, alkoxy, haloalkoxy, alkylthio, haloalkylthio, CN, aryl, arylalkyl, aryloxy, arylalkoxy, arylalkylthio, alkylene, haloalkylene, alkylenedioxy, haloalkylenedioxy, wherein said aryl, arylalkyl, aryloxy, arylalkoxy, arylalkylthio is optionally substituted with independently selected halogen, alkyl, haloalkyl, alkoxy, or haloalkoxy;
X is a bridge consisting of 2 to 8 carbon atoms, 0 to 1 nitrogen atom, 0 to 1 oxygen atom, and 0 to 1 sulfur atom optionally substituted with halogen, alkyl, haloalkyl, hydroxy, alkoxy, haloalkoxy, alkylthio, cyano, acyl, alkylsulfinyl, alkylsulfonyl, or NO₂
n is 1 or 2; or a salt thereof.

2. The compound of claim 1 wherein R₁ and R₂ are independently selected from the group consisting of alkyl, COR₆, COOR₆, CONR₇R₈, SO₂R₉, aryl or arylalkyl optionally substituted with halogen, alkyl, haloalkyl, alkoxy, haloalkoxy, alkylenedioxy, or haloalkylenedioxy.

3. The compound of claim 1 wherein R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring.

4. The compound of claim 1 wherein R₃ and R4 are each independently selected from the group consisting of separately from alkyl, arylalkyl or heteroarylalkyl optionally substituted with halogen, alkyl, haloalkyl, alkoxy, haloalkoxy, alkylenedioxy, or haloalkylenedioxy.

5. The compound of claim 1 wherein R₃ and R₄ together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring.

6. The compound of claim 1 wherein R₅ is a non-bonded pair of electrons, or is selected from the group consisting of alkyl, arylalkyl or heteroarylalkyl optionally substituted with halogen, alkyl, haloalkyl, alkoxy, haloalkoxy, alkylenedioxy, or haloalkylenedioxy.

7. The compound of claim 1 wherein when R₅ is selected from the group consisting of alkyl, arylalkyl or heteroarylalkyl optionally substituted with halogen, alkyl, haloalkyl, alkoxy, haloalkoxy, alkylenedioxy, or haloalkylenedioxy, the N-atom to which R₃, R₄, and R₅ are attached will carry a positive charge and be associated with anion Y⁻, where Y may be halogen, carboxylate, sulfonate or any other suitable counterion.

8. The compound of claim 1 wherein R₆, R₇, R₈ and R₉ are each independently selected from the group consisting of H, alkyl, haloalkyl, alkoxyalkyl, haloalkoxyalkyl, alkylthioalkyl, haloalkylthioalkyl, aryl or arylalkyl optionally substituted with halogen, alkyl, haloalkyl, alkoxy, haloalkoxy, alkylthio, haloalkylthio, alkylene, haloalkylene, alkylenedioxy, haloalkylenedioxy, CN, aryl, arylalkyl, aryloxy, arylalkoxy, arylalkylthio wherein said aryl, arylalkyl, aryloxy, arylalkoxy, arylalkylthio is optionally substituted with independently selected halogen, alkyl, haloalkyl, alkoxy, or haloalkoxy.

9. The compound of claim 1 wherein X is a bridge consisting of 2 to 8 carbon atoms, 0 to 1 nitrogen atom, 0 to 1 oxygen atom, and 0 to 1 sulfur atom, wherein said bridge is optionally substituted with halogen, alkyl, haloalkyl, hydroxy, alkoxy, or haloalkoxy.

10. The compound of claim 1 wherein R₁ is taken from COR₆ or SO₂R₉, and R₂ is arylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂.

11. The compound of claim 10 wherein R₁ is taken from COR₆, and R₂ is taken from arylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂.

12. The compound of claim 10 wherein R₁ is taken from SO₂R₉, and R₂ is taken from arylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂.

13. The compound of claim 10 wherein R₆ and R₉ are taken from aryl optionally substituted with halogen, alkyl, haloalkyl, alkoxy, haloalkoxy, alkylthio, haloalkylthio, alkylenedioxy, haloalkylenedioxy, or CN.

14. The compound of claim 11 wherein R₆ is aryl optionally substituted with halogen, alkyl, haloalkyl, alkoxy, haloalkoxy, alkylthio, haloalkylthio, alkylenedioxy, haloalkylenedioxy, or CN.

15. The compound of claim 12 wherein R₉ is aryl optionally substituted with halogen, alkyl, haloalkyl, alkoxy, haloalkoxy, alkylthio, haloalkylthio, alkylenedioxy, haloalkylenedioxy, or CN.

16. The compound of claim 10, wherein R² is benzyl or naphthyl optionally substituted with halogen, alkyl, haloalkyl, alkoxy, haloalkoxy, alkylthio, cyano, acyl, alkylsulfinyl, alkylsulfonyl, alkylenedioxy, haloalkylenedioxy, or NO₂.

17. The compound of claim 11, wherein R⁶ is taken from phenyl, naphthyl, or 2-benzothienyl optionally substituted with halogen, alkyl, haloalkyl, alkoxy, haloalkoxy, alkylthio, cyano, acyl, alkylsulfinyl, alkylsulfonyl, alkylenedioxy, haloalkylenedioxy, or NO₂.

18. The compound of claim 12, wherein R⁹ is taken from phenyl or naphthyl optionally substituted with halogen, alkyl, haloalkyl, alkoxy, haloalkoxy, alkylthio, cyano, acyl, alkylsulfinyl, alkylsulfonyl, alkylenedioxy, haloalkylenedioxy, or NO₂.

19. The compound of claim 1, wherein R¹ is taken from COR⁶; R² is taken from arylalkyl optionally substituted with halogen, alkyl, haloalkyl, alkoxy, haloalkoxy, alkylthio, cyano, acyl,alkylsulfinyl, alkylsulfonyl, alkylenedioxy, haloalkylenedioxy, or NO₂; R³ and R⁴ are taken from lower alkyl; R⁵ is taken from arylalkyl optionally substituted with halogen, alkyl, haloalkyl, alkoxy, haloalkoxy, alkylthio, cyano, acyl, alkylsulfinyl, alkylsulfonyl, alkylenedioxy, haloalkylenedioxy or NO₂; and X is alkylene.

20. The compound of claim 1, wherein R¹ is taken from SO₂R⁹; R² is taken from arylalkyl optionally substituted with halogen, alkyl, haloalkyl, alkoxy, haloalkoxy, alkylthio, cyano, acyl,alkylsulfinyl, alkylsulfonyl, alkylenedioxy, haloalkylenedioxy, or NO₂; R³ and R⁴ are taken from lower alkyl; R⁵ is taken from arylalkyl optionally substituted with halogen, alkyl, haloalkyl, alkoxy, haloalkoxy, alkylthio, cyano, acyl, alkylsulfinyl, alkylsulfonyl, alkylenedioxy, haloalkylenedioxy, or NO₂; and X is alkylene.

21. A composition for controlling and preventing plant pathogenic microorganisms comprising, in combination, a compound of claim 1 together with a suitable carrier.

22. The composition of claim 21, further comprising at least one additional fungicide or systemically acquired resistance inducer.

23. A method of controlling or preventing infestation of cultivated plants by pathogenic microorganisms, comprising:
applying a compound according to claim 1 to said plants, parts thereof or the locus thereof in an amount effective to control said microorganisms.

24. A method according to claim 23, wherein the microorganism is a fungal organism.

25. The method of claim 24, wherein said fungal organism is selected from the group consisting of Septoria tritici, Stagonospora nodorum, Phytophthora infestans, Botrytis cinerea, Sclerotinia homoeocarpa and Puccinia recondita.

26. A method of controlling or preventing infestation of plant propagation material by pathogenic microorganisms, comprising:
applying a compound according to claim 1 to said plant propagation material in an amount effective to control said microorganisms.

27. The method of claim 26, wherein said plant propagation material comprises seeds.

28. A method according to claim 26, wherein the microorganism is a fungal organism.

29. A method of controlling or preventing infestation of a technical material by pathogenic microorganisms, comprising:
applying a compound according to claim 1 to said technical material in an amount effective to control said microorganisms.

30. A method of treating a fungal infection in a subject in need thereof, comprising:
administering a compound of claim 1 or a pharmaceutically acceptable salt thereof to said subject in an amount effective to treat said fungal infection.

31. A composition for treating a fungal infection in a subject in need thereof, comprising, in combination, a compound of claim 1 or a pharmaceutically acceptable salt thereof together with a pharmaceutically acceptable carrier.

32. A method for treating obesity, an overweight condition, hypertriglyceridemia, hyperlipidemia, hypoalphalipoproteinemia, Metabolic Syndrome, diabetes mellitus, hyperinsulinemia, impaired glucose tolerance, insulin resistance, diabetic complications, atherosclerosis, hypertension, coronary heart disease, hypercholesterolemia, stroke, polycystic ovary disease, cerebrovascular disease or congestive heart failure in a mammal by administering to a mammal in need of such treatment a therapeutically effective amount of a compound of claim 1, a prodrug thereof, or a pharmaceutically acceptable salt of said compound or of said prodrug.

33. A method according to claim 32 wherein atherosclerosis is treated.

34. A method according to claim 32 wherein diabetes is treated.

35. A method according to claim 32 wherein obesity is treated.

36. A pharmaceutical composition which comprises in combination a compound of claim 1, a prodrug thereof, or a pharmaceutically acceptable salt of said compound or of said prodrug and a pharmaceutically acceptable carrier, vehicle or diluent.

37. A pharmaceutical combination composition comprising in combination: a therapeutically effective amount of a composition comprising a first compound, said first compound being a formula I compound, a prodrug thereof, or a pharmaceutically acceptable salt of said compound or of said prodrug; a second compound, said second compound being an anti-atherosclerosis agent, an anti-diabetic agent, an anti-obesity agent or a cardiovascular agent and/or optionally a pharmaceutically acceptable vehicle, diluent or carrier.

38. A kit for achieving a therapeutic effect in a mammal which has been prescribed the joint administration of the active ingredients as (a) and (b) below, each active ingredient forming a portion of said kit, comprising in combination:
(a) a first compound, said first compound being a compound of claim 1, a prodrug thereof, or a pharmaceutically acceptable salt of said compound or of said prodrug and a pharmaceutically acceptable carrier, vehicle or diluent in a first unit dosage form;
(b) a second compound, said second compound being an anti-atherosclerosis agent, an anti-diabetic agent, an anti-obesity agent or a cardiovascular agent and a pharmaceutically acceptable vehicle, diluent or carrier in a second unit dosage form; and
(c) directions for the administration of active ingredients (a) and (b) in a manner to achieve a desired therapeutic effect and wherein the amounts of the first and second compounds result in a therapeutic effect.

39. A method of making a compound of formula I: 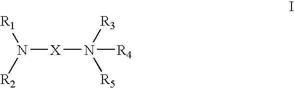
wherein
R₁ and R₂ are taken independently from H, alkyl, alkenyl, alkynyl, haloalkyl, haloalkenyl, haloalkynyl, COR₆, COOR₆, CONR₇R₈, SO₂NR₇R₈, SO₂R₉, alkoxyalkyl, alkylthioalkyl, aryl, arylalkyl or aryloxyalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,
or R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring;
R₃ and R₄ are taken separately from alkyl, alkenyl, alkynyl, haloalkyl, haloalkenyl, haloalkynyl, CH₂COR₆, CH₂COOR₆, CH₂CONR₇R₈, O, aryl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,
or R₃ and R₄ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═(O)ₙ, ═S, and optionally fused to another 5- or 6-membered ring;
R₅ may be a non-bonded pair of electrons, alkyl, alkenyl, alkynyl, haloalkyl, haloalkenyl, haloalkynyl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂;
when R₅ is alkyl, alkenyl, alkynyl, haloalkyl, haloalkenyl, haloalkynyl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂, the N-atom to which R₃, R₄, and R₅ are attached will carry a positive charge and be associated with anion Y⁻, where Y may be halogen, carboxylate, sulfonate or any other suitable counterion;
R₆, R₇, R₈ and R₉ are each independently selected from the group consisting of H, alkyl, haloalkyl, alkoxyalkyl, haloalkoxyalkyl, alkylthioalkyl, haloalkylthioalkyl, aryl or arylalkyl optionally substituted with halogen, alkyl, haloalkyl, alkoxy, haloalkoxy, alkylthio, haloalkylthio, CN, aryl, arylalkyl, aryloxy, arylalkoxy, arylalkylthio, alkylene, haloalkylene, alkylenedioxy, haloalkylenedioxy, or wherein said aryl, arylalkyl, aryloxy, arylalkoxy, arylalkylthio is optionally substituted with independently selected halogen, alkyl, haloalkyl, alkoxy, or haloalkoxy;
X is a bridge group consisting of 2 to 8 C-atoms, 0 to 1 N-atom, 0 to 1 O-atom and 0 to 1 S-atom optionally substituted with halogen, alkyl, haloalkyl, hydroxy, alkoxy, haloalkoxy, alkylthio, cyano, acyl, alkylsulfinyl, alkylsulfonyl, or NO₂
n is 1 or 2;
comprising:
alkylation of an amine II with an appropriate halide (Q=Cl, Br, or I) in an inert solvent.

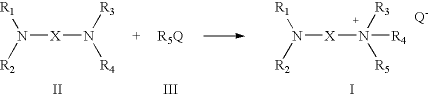

40. A method of making a compound of formula I: 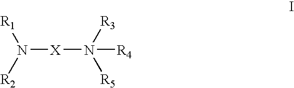
wherein
R₁ and R₂ are taken independently from H, alkyl, alkenyl, alkynyl, haloalkyl, haloalkenyl, haloalkynyl, COR₆, COOR₆, CONR₇R₈, SO₂NR₇R₈, SO₂R₉, alkoxyalkyl, alkylthioalkyl, aryl, arylalkyl or aryloxyalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,
or R₁ and R₂ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═O, ═S, and optionally fused to another 5- or 6-membered ring;
R₃ and R₄ are taken separately from alkyl, alkenyl, alkynyl, haloalkyl, haloalkenyl, haloalkynyl, CH₂COR₆, CH₂COOR₆, CH₂CONR₇R₈, aryl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂,
or R₃ and R₄ may be taken together to form a 5- or 6-membered ring comprised of 3 to 5 carbon atoms, 1 to 2 nitrogen atoms, 0 to 1 oxygen atom, and 0 to 1 sulfur atom, optionally substituted by halogen, alkyl, haloalkyl, alkoxyalkyl, haloalkoxyalkyl, arylalkyl, ═(O)ₙ, ═S, and optionally fused to another 5- or 6-membered ring;
R₅ may be a non-bonded pair of electrons, alkyl, alkenyl, alkynyl, haloalkyl, haloalkenyl, haloalkynyl, arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂;
when R₅ is alkyl, alkenyl, alkynyl, haloalkyl, haloalkenyl, haloalkynyl; arylalkyl or heteroarylalkyl optionally substituted with halogen, alkyl, haloalkyl, alkoxy, haloalkoxy, alkylthio, cyano, acyl, alkylsulfinyl, alkylsulfonyl, alkylene, haloalkylene, alkylenedioxy, haloalkylenedioxy, or NO₂, the N-atom to which R₃, R₄, and R₅ are attached will carry a positive charge and be associated with anion Y⁻, where Y may be halogen, carboxylate, sulfonate or any other suitable counterion;
R₆, R₇, R₈ and R₉ are each independently selected from the group consisting of H, alkyl, haloalkyl, alkoxyalkyl, haloalkoxyalkyl, alkylthioalkyl, haloalkylthioalkyl, aryl or arylalkyl optionally substituted with halogen, alkyl, haloalkyl, alkoxy, haloalkoxy, alkylthio, haloalkylthio, CN, aryl, arylalkyl, aryloxy, arylalkoxy, arylalkylthio wherein said aryl, arylalkyl, aryloxy, arylalkoxy, arylalkylthio, alkylene, haloalkylene, alkylenedioxy, haloalkylenedioxy is optionally substituted with independently selected halogen, alkyl, haloalkyl, alkoxy, or haloalkoxy;
X is a bridge group consisting of 2 to 8 C-atoms, 0 to 1 N-atom, 0 to 1 O-atom and 0 to 1 S-atom optionally substituted with halogen, alkyl, haloalkyl, hydroxy, alkoxy, haloalkoxy, alkylthio, cyano, acyl, alkylsulfinyl, alkylsulfonyl, or NO₂
n is 1 or 2;
comprising:
alkylation of an amine II with a sulfonate ester (Q=OSO₂R′) III in an inert solvent. [chemical structure]
